(19) United States
(12) Patent Application Publication
(10) Pub. No.: US 2022/0387727 A1
(43) Pub. Date: Dec. 8, 2022

(54) NEEDLE ADAPTOR AND ASSEMBLY FOR FORMING AN INJECTION DEVICE FOR ADMINISTERING A FLUID TO A SUBJECT

(71) Applicant: NOVOSANIS NV, Wijnegem (BE)

(72) Inventors: Koen Catharina Lodewijk BEYERS, Wuustwezel (BE); Timothi Julio Steven VAN MULDER, Schilde (BE); Nette MEERS, Lier (BE); Alejandra R#OS CORTÉS, Kortenberg (BE); Quinten VAN AVONDT, Lier (BE); Vanessa Vicky Jill VANKERCKHOVEN, Wilrijk (BE)

(73) Assignee: IDEVAX, Antwerpen (BE)

(21) Appl. No.: 17/770,006

(22) PCT Filed: Oct. 16, 2020
(86) PCT No.: PCT/EP2020/079267

§ 371 Date: Apr. 18, 2022

Related U.S. Application Data
(60) Provisional application No. 62/916,157, filed on Oct. 16, 2019.

(51) Int. Cl. A61M 5/32; A61M 25/06

(52) U.S. Cl. CPC A61M 5/32; A61M 25/0625; A61M 2005/325

(57) ABSTRACT

A needle adaptor for forming an injection device for administering a fluid to a subject is disclosed. An assembly for forming an injection device for administering a fluid to a subject comprising a needle adaptor body is also disclosed. Methods for assembling the needle adaptor and the assembly are further disclosed, as are methods of administering a fluid to a subject using same, and kits and injection devices comprising same.

### FIELD OF THE INVENTION

The present invention pertains to a needle adaptor as well as an assembly for forming an injection device for administering a fluid to a subject. The invention also pertains to methods for assembling the needle adaptor and the assembly, methods of administering a fluid to a subject using same, and kits and injection devices comprising same.

### BACKGROUND

A wide variety of injection devices are known in the art, the most well-known of which is a classical plastic medical syringe, fitted with a detachable stainless steel needle. Such syringes are used to deliver active agents such as drugs and vaccines via various administration routes requiring different injection depths, such as, for example, intradermal (ID), intravenous (IV), subcutaneous (SC), or intramuscular (IM) injections. While classical plastic medical syringes are relatively cheap to manufacture by virtue of their simple mechanical structure, they do not have any built-in functionality to assist with controlled penetration of the skin to a predefined depth. As such, correct use of classical syringes for the above-noted administration routes is reliant on the skills of the person administering the active agent.

Morbidity and mortality due to infectious diseases have been dramatically reduced by vaccination, which is the most cost-effective public health measure to prevent the spread of disease (Lambert et al., Can successful vaccines teach us how to induce efficient protective immune responses? Nature Medicine. 2005: 11: S54-S62). Three main routes of vaccine administration include ID injection, SC injection and IM injection. Interestingly, most vaccines are given by IM injection, even though the muscle is not a highly immunogenic organ (Hutin et al., Use of injections in healthcare settings worldwide, 2000: literature review and regional estimates. British Medical Journal. 2003; 327:1075-1078; Hohlfed and Engel, The immunobiology of muscle. Immunology Today. 1994; 15: 269-274). The skin, in contrast, is a much more attractive site for vaccination because of the large number of resident dendritic cells and efficient drainage to lymph nodes (Debenedictis et al., Immune functions of the skin. Clinics in Dermatology. 2001; 19:573-585; Kupper and Fuhlbrigge, Immune surveillance in the skin: mechanisms and clinical consequences. Nature Reviews Immunology. 2004; 4:211-222), with the result that smaller doses of antigen might induce an equivalent immune response to the standard dose. Antigen trafficking studies have shown that ID vaccination leads to more efficient antigen migration into lymph nodes than conventional IM delivery (Steinman and Branchereau, Taking dendritic cells into medicine. Nature. 2007; 449: 419-426; Valladeau and Saeland, Cutaneous dendritic cells. Seminars in Immunology. 2005; 17: 273-283; Sugita et al, Innate immunity mediated by epidermal keratinocytes promotes acquired immunity involving Langerhans cells and T cells in the skin. Clinical and Experimental Immunology. 2007; 147: 176-183). Skin vaccinations, however, have not been widely adopted because ID injection requires specialized training and, even with training, does not reliably target the skin (Flynn et al., Influence of needle gauge in Mantoux skin testing. Chest. 1994; 106:1463-1465). Today, most ID injections are delivered by specially trained personnel with a conventional hypodermic needle, via the Mantoux technique. The needle must be inserted into the skin at a 5 to 15 degree angle. Difficulties associated with performing this injection into the skin have historically limited its use, even though fractional doses of some vaccines are effective when injected in the skin.

The skin, as the primary interface between the body and the environment, provides the first line of defence against a broad array of microbial pathogens (Debenedictis et al., Immune functions of the skin. Clinics in Dermatology. 2001; 19:573-585; Kupper and Fuhlbrigge, Immune surveillance in the skin: mechanisms and clinical consequences. Nature Reviews Immunology. 2004; 4:211-222). Although skin-targeted immunization has been utilized for decades, its application beyond a few vaccines has been hindered by the lack of simple and reliable skin vaccination technology. An alternative method for ID injection is ID microinjection. Skin vaccination with microneedles has the potential to improve both the immunology and logistics of vaccination. Compared to IM injections, skin vaccinations with microneedles eliminate or reduce the pain and apprehension felt by patients, eliminate or reduce the risk of needle-stick injury, and enable increased vaccination coverage, since skin vaccines can be administered by minimally trained medical professionals or by the patient themselves.

The need for safe, economic and efficient vaccine administration and the increasing mechanistic knowledge of immune responses induced by targeting the ID layers of the skin have all driven the engineering of novel delivery devices for ID injection (Wang et al., Precise microinjection into skin using hollow microneedles. 2006; 126: 1080-1087; Kim and Prausnitz, Enabling skin vaccination using new delivery technologies. Drug Delivery and Translational Research. 2011; 1(1):7-12). Specifically, these advanced delivery technologies employ microneedles that are inserted 1.5 mm perpendicularly into the skin and which inject approximately 100-200 μL of a liquid vaccine into the dermal skin layers. There are promising clinical data with some vaccines that highlight the potential of reduced-dose immunization via this ID route (Zehrung et al., Intradermal delivery for vaccine dose sparing: overview of current issues. Vaccine. 2013; 31(34): 3392-3395). ID injections have the potential to increase vaccine effectiveness in specific populations and may help to increase vaccine access, reduce costs, and ease the logistical burdens of immunization programs, especially in low-resource settings.

New devices for easier, more reliable ID delivery are being developed that may serve as alternatives to the Mantoux technique and help to promote the implementation of dose-sparing ID vaccination strategies. The range of new devices for ID delivery include adapters for traditional needles and syringes that control the depth and angle of needle penetration, mini-needles, microneedles, and ID liquid jet injectors (Zehrung et al., Intradermal delivery for vaccine dose sparing: overview of current issues. Vaccine. 2013; 31(34): 3392-3395). Most of these devices are currently only available for research purposes.

A highly-sophisticated injection device is described in WO2013156524(A1). It contains a foot to be placed on a skin, a double-ended moveable needle, and a reservoir or a container containing a fluid to be administered. The device has a highly sophisticated mechanism to guarantee a specific sequence of events. First, the device needs to be unlocked. Then, one first end of the needle enters the reservoir. Then, the reservoir and needle move inside the device and a second end of the needle penetrates the skin. In other words, a double-pointed needle will on the one side enter a prefilled reservoir, and on the other side penetrate the skin. Subsequently, the reservoir is emptied by pushing down the plunger, and finally the needle is retracted. This device is ideally suited for ID injections.

Another highly-sophisticated assembly for forming an injection device is described in WO2017168015(A1). The assembly includes a foot to be placed on a skin; a body comprising at least one needle, wherein the body is movably mounted to the foot for allowing movement of the needle towards the skin. The needle extends out of a second contact surface by a predefined distance for limiting a penetration depth of the needle. The assembly further includes a first friction means for preventing movement of the body relative to the foot for causing a sudden acceleration, and a second friction means for creating a dynamic friction when the needle is moving towards the skin for keeping the skin stretched. The assembly is particularly suitable for ID injections, although it can also be used for IV, SC, or IM injections in certain embodiments.

There is a need for new injection devices, in particular for those suited to ID injections.

This background information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

In one aspect, there is provided a needle adaptor for forming an injection device for administering a fluid to a subject comprising a housing formed from a first housing portion and a second housing portion, the housing having a proximal end and a distal end; and a needle unit fixedly mounted within the housing. The needle unit comprises a needle shaft comprising a first end for penetrating the subject's skin and a second end connected to a needle hub, the needle hub comprising a distal end connected to the second end of the needle shaft and a proximal end comprising a pair of radially extending diametrically opposing flanges. Each of the first housing portion and the second housing portion comprises at least two consecutive transverse walls or projections extending from an inner surface thereof, wherein the at least two consecutive transverse walls or projections form a gap therebetween for receiving at least a portion of one or both of the pair of radially extending diametrically opposing flanges of the needle unit to fixedly mount the needle unit within the housing. The proximal end of the housing together with the at least two consecutive transverse walls or projections of each of the first housing portion and the second housing portion define a channel for receiving a syringe tip for engagement with the needle hub. The distal end of the housing comprises a first contact surface adapted to be placed on a skin of the subject and a second contact surface, wherein the first end of the needle shaft extends out of the second contact surface by a predefined distance for limiting a penetration depth of the needle shaft.

In another aspect, there is provided an assembly for forming an injection device for administering a fluid to a subject, the assembly comprising a foot comprising a first contact surface adapted to be placed on a skin of the subject, the foot having a tubular shape for receiving a needle adaptor body, and a needle adaptor body. The needle adaptor body comprises a housing formed from a first housing portion and a second housing portion, the housing having a proximal end and a distal end; and a needle unit fixedly mounted within the housing. The needle unit comprises a needle shaft comprising a first end for penetrating the subject's skin and a second end connected to a needle hub, the needle hub comprising a distal end connected to the second end of the needle shaft and a proximal end comprising a pair of radially extending diametrically opposing flanges. Each of the first housing portion and the second housing portion comprises at least two consecutive transverse walls or projections extending from an inner surface thereof, wherein the at least two consecutive transverse walls or projections form a gap therebetween for receiving at least a portion of one or both of the pair of radially extending diametrically opposing flanges of the needle unit to fixedly mount the needle unit within the housing. The proximal end of the housing together with the at least two consecutive transverse walls or projections of each of the first housing portion and the second housing portion define a channel for receiving a syringe tip for engagement with the needle hub. The distal end of the housing comprises a second contact surface, wherein the first end of the needle shaft extends out of the second contact surface by a predefined distance for limiting a penetration depth of the needle shaft. The needle adaptor body is movably mounted to the foot for allowing movement of the needle adaptor body from a first position to a second position, wherein when the needle adaptor body is in the first position, the needle shaft is in a retracted position such that the first end of the needle shaft does not extend beyond the first contact surface, and when the needle adaptor body is in the second position, the first end of the needle shaft extends beyond the first contact surface and out of the second contact surface by the predefined distance for limiting the penetration depth of the needle shaft. The assembly further comprises a friction means for inhibiting movement of the needle adaptor body relative to the foot when the needle adaptor body is in the first position, until a predefined static friction force is overcome, and for causing or allowing a sudden acceleration of the needle adaptor body towards the foot for increasing a speed of the needle shaft for increasing chance of penetration of the skin.

### BRIEF DESCRIPTION OF THE FIGURES

For a better understanding of the present invention including the progression of development to get to the end product, reference is made to the following description which is to be used in conjunction with the accompanying drawings, where:

FIG. 1(a) illustrates an exemplary needle adaptor according to an embodiment of the present application, in perspective view.

FIG. 1(b) illustrates a top view of the needle adaptor shown in FIG. 1(a), showing the proximal end of the housing and the channel for receiving a syringe tip formed therein.

FIG. 1(c) illustrates a bottom view of the needle adaptor 100 shown in FIG. 1(a), showing the distal end 106 of the housing 102 comprising a first contact surface 132 adapted to be placed on a skin of the subject, and a second contact surface 134 through which the first end 112 of the needle shaft 110 extends.

FIGS. 2(a) and 2(b) illustrate the first housing portion 102a and the manner in which the needle unit 108 can be engaged therewith.

FIG. 3(a) illustrates the partially disassembled needle adaptor 100 of FIG. 1(a), showing the first housing portion 102a, the second housing portion 102b, and the needle unit 108 engaged with the first housing portion 102a in the first orientation. FIG. 3(b) illustrates the partially disassembled needle adaptor 100 of FIG. 1(a), showing the first housing portion 102a, the second housing portion 102b, and the needle unit 108 engaged with the first housing portion 102a in the second orientation.

FIGS. 4(a) and 4(b) illustrate a simplified first housing portion 102a of the needle adaptor 100 wherein the distal end of the housing 106 lacks the first contact surface 132, in order to better illustrate how varying the placement of the needle unit 108 within the first housing portion 102a impacts the predefined distance d1 that the first end of the needle shaft 112 extends out of the second contact surface 134.

FIG. 5 represents a further simplified view of the simplified first housing portion 102a shown in FIGS. 4(a) and 4(b), and also represents a simplified first housing position 202a of the needle adaptor body 200 of the assembly 201 discussed in further detail below. In the labels for FIG. 5, references to components/elements of the needle adaptor body 200 of the assembly 201 discussed below are provided in brackets.

FIG. 6 illustrates a perspective view of the first housing portion 102a, showing how a preselected portion p1 of a distal end of the first housing portion 102a can be removed during assembly of the needle adaptor 100 to further account for manufacturer variability in needle shaft lengths.

FIGS. 7(a) and (b) show a simplified cross-sectional view of an embodiment of the needle adaptor 100 engaged with a syringe 146, thus forming an injection device 149 for administering a fluid to a subject via injection. FIG. 7(c) illustrates a perspective view of the injection device 149 shown in FIGS. 7(a) and (b).

FIG. 8 illustrates a series of steps that can be used in administering a fluid to a subject via injection using the needle adaptor 100.

FIG. 9(a) illustrates the safety holder 152. FIG. 9(b) illustrates a simplified cross-sectional view of an embodiment of the needle adaptor 100 engaged with a syringe 146, and how the distal end 106 of the needle adaptor housing 102 can be received in the open end 154 of the safety holder. FIG. 9(c) illustrates the needle adaptor housing 102 engaged with the safety holder 152, with the needle adaptor 100 in a simplified cross-sectional view. FIG. 9(d) illustrates the needle adaptor housing 102 engaged with the safety holder 152, with the needle adaptor 100 in a simplified cross-sectional view and with the safety holder 152 and syringe 146 also in cross-sectional view.

FIG. 10 illustrates the engagement of a syringe 146 with a dose-metering device 160, and the engagement of same with the needle adaptor 100.

FIG. 11(a) illustrates an exemplary assembly 201 for forming an injection device for administering a fluid to a subject according to an embodiment of the present application, in perspective view.

FIG. 11(b) illustrates a top view of the assembly shown in FIG. 11(a), showing the proximal end of the needle adaptor housing and the channel for receiving a syringe tip formed therein.

FIG. 11(c) illustrates a further perspective view of the assembly shown in FIG. 11(a).

FIG. 11(d) illustrates a bottom view of the assembly 201 shown in FIGS. 11(a) and (c), showing the distal end 233 of the foot 231 comprising a first contact surface 232 adapted to be placed on a skin of the subject. The second contact surface 234 of the distal end 206 of the housing 202 of the needle adaptor body 200 through which the first end 112 of the needle shaft 210 extends is also visible through an aperture 286 formed by an interior surface 288 of the foot, the interior surface of the foot being oriented in a plane substantially parallel to and spaced from a tangential plane defined by the first contact surface 232.

FIG. 12 illustrates an exploded view of the assembly 201 shown in FIGS. 11(a) and (c).

FIGS. 13(a) and 13(b) illustrate the first housing portion 202a and the manner in which the needle unit 208 can be engaged therewith.

FIGS. 14(a)-(c) illustrate engagement of the needle unit 208 with the first housing portion 202a, and engagement of the first housing portion 202a with the second housing portion 202b to form the housing 202 of the needle adaptor body 200.

FIG. 15 illustrates a perspective view of the needle adaptor body 200 and foot 231, where the at least two protrusions 274 extending from an inner surface 276 of a proximal end 278 of the foot 231 and one of the at least two corresponding grooves 280 located on an outer surface 266 of the distal end 206 of the housing 202 of the needle adaptor body 200 can be clearly seen.

FIG. 16 provides an enlarged perspective view of the needle adaptor body 200 to better illustrate the contours of groove 280.

FIG. 17(a) illustrates an embodiment of an assembly 201 in perspective (upper) and cross-sectional view (lower) wherein the safety clip 264 has been removed and the needle adaptor body 200 is in the first position (i.e. ready for injection). FIG. 17(b) illustrates assembly 201 in perspective (upper) and cross-sectional view (lower) wherein the safety clip 264 has been removed and the needle adaptor body 200 is in the second position (i.e. the needle shaft 210 penetrates the skin). FIG. 17(c) illustrates assembly 201 in perspective (upper) and cross-sectional view (lower) wherein the needle adaptor body 200 is held in a fixed, deactivated position relative to the foot 231.

FIG. 18(a) illustrates a proposed automatic assembly line for preparing assembly 201 using Machine Vision and pick-and-place robotics technology. “Housing 1” and “Housing 2” refer to first and second housing portions 202a and 202b, “Needle” refers to needle unit 208, “Housing Mounting” refers to mounting needle unit 208 in one of the first and second housing portions 202a/202b, “Pull Pin” refers to the safety clip 264, and “Foot” refers to foot 231. The various elements are placed in the production carrier which moves along the assembly line via a conveyer belt. FIG. 18(b) illustrates the production carrier makeup at each stage of the assembly.

FIG. 19 illustrates a series of steps that can be used in administering a fluid to a subject via injection using the assembly 201.

FIGS. 20(a) and (b) show a simplified cross-sectional view of an alternate embodiment of the assembly 201 engaged with a syringe 246, thus forming an injection device 249 for administering a fluid to a subject via injection. As can be seen from FIGS. 20(a) and (b), the locking mechanism is absent and the needle adaptor body 200 is in the second position wherein the needle shaft 210 penetrates the skin 250 (shown in FIG. 20(b)). FIG. 20(c) illustrates a perspective view of the injection device 249 shown in FIGS. 20(a) and (b).

### DETAILED DESCRIPTION OF ILLUSTRATIVE EMBODIMENTS

### Definitions

Unless defined otherwise, all technical and scientific terms used herein have the same meaning as commonly understood by one of ordinary skill in the art to which this invention belongs.

As used in the specification and claims, the singular forms “a”, “an” and “the” include plural references unless the context clearly dictates otherwise.

The term “comprising” as used herein will be understood to mean that the list following is non-exhaustive and may or may not include any other additional suitable items, for example one or more further feature(s), component(s) ingredient(s) and/or elements(s) as appropriate.

Terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

The term “fluid” as used herein will be understood to mean any matter which can be injected through a needle, such as for example a liquid, a solution, a suspension, a gel, or other substances which can be injected via a needle.

The terms first, second and the like in the present specification are used in order to distinguish between similar elements, and it will be understood that these terms may be interchangeable under certain circumstances. In addition, the terms top, bottom, etc. in the present specification are used for descriptive purposes and may not denote relative positions. Particular features of one or more embodiments of the present application may be combined in any suitable manner, as would be understood by a skilled worker in view of the teaching of the present application. Finally, the drawings provided herein are for illustrative purposes and elements illustrated therein may not be drawn to scale. In the drawings, like reference numerals refer to like parts throughout the various views and as described herein, unless otherwise specified.

Current state-of-the-art needle adaptors and injection devices consist of a needle unit or an array of needle units which are mounted, such as by gluing or overmoulding, in an adaptor piece or other device. Such needle units typically comprise a stainless steel needle shaft which may be in a plastic (e.g. polypropylene (PP)), metal or potentially even glass hub.

With respect to overmoulding: in this case, a needle unit/shaft is positioned in an injection moulding tool in a specific purpose built cavity (designed to keep the needle tip and part of the shaft free of plastic). Subsequently, Medical Grade plastic (e.g. Cyclic Olefin Copolymer (COC)) would be overmoulded, making a firm connection between needle shaft and hub/housing. The downsides to overmoulding are that the process is difficult to automate (and yet when not heavily automated, the process is very expensive), requires complex tooling, is particularly challenging when very short needle shafts are needed, is heavily depending on needle accuracy and tolerances, and the needle tip can be damaged during the process.

With respect to gluing: a needle shaft can be positioned and mounted in a (e.g. injection moulded) hub or housing by means of glue, which would require a (semi)-automated system to hold the needle shaft, hold the housing, position the 2 components with respect to each other, and mount the needle shaft with e.g. ultra-violet (UV) curing glue or silicone. The downsides to gluing are that it can present biocompatibility issues (where elements of the glue may be extracted/leached into fluids to be injected, etc.), poses quality control issues (with respect to positioning, leakage of the device, etc.), may be subject to creep in needle shaft/unit positioning over time (which may affect needle shaft length for injections), is very difficult for short needle shafts, and is expensive to automate.

In addition, current state-of-the-art needle adaptors and injection devices claim to have a pre-defined needle protrusion length of e.g. 1 mm or e.g. 13 mm. However, as a result of current state-of-the-art manufacturing processes, it is known that final needle lengths will be subject to production tolerances of e.g. 0.05 mm, or e.g. 2 mm as defined in specific ISO standards.

As such, it will be understood that the current state-of-the-art is generally preferred for long needles (e.g. +5 mm) having broad tolerances (e.g. +:−0.5 mm), as only then it is inexpensive (due to applied dimensions and tolerances).

In view of the foregoing, it will be understood that current technology can fall short when shallow penetration depths are required (requiring a shorter functional needle shaft length for injection, such as in the case of ID injections), as it is inaccurate, expensive, and error-prone.

The needle adaptor and assembly for forming an injection device for administering a fluid to a subject described herein address the above-noted deficiencies, and allow for control over penetration depth regardless of intended needle length and tolerance deviations. The needle adaptor and assembly of the present application allow for the use of needle units having longer needle shafts, such as (pre-glued) commercially available needle units comprising a needle shaft and hub having a standard female Luer-Lok fitting, 26-34 G and 12 mm length. Such needle units can have long needle shafts with broad tolerances, while the needle adaptor and assembly of the present application can accurately control penetration depth irrespective of same. The needle adaptor and assembly of the present application can therefore account for and offset manufacturer variability in needle shafts.

In one embodiment of the present application, there is provided a needle adaptor for forming an injection device for administering a fluid to a subject comprising a housing formed from a first housing portion and a second housing portion, the housing having a proximal end and a distal end; and a needle unit fixedly mounted within the housing. The needle unit comprises a needle shaft comprising a first end for penetrating the subject's skin and a second end connected to a needle hub, the needle hub comprising a distal end connected to the second end of the needle shaft and a proximal end comprising a pair of radially extending diametrically opposing flanges (e.g. typical needle hub tabs that would be found on commercially available needle units comprising a needle shaft and hub having a standard female Luer-Lok fitting). Each of the first housing portion and the second housing portion comprises at least two consecutive transverse walls or projections extending from an inner surface thereof, wherein the at least two consecutive transverse walls or projections form a gap therebetween for receiving at least a portion of one or both of the pair of radially extending diametrically opposing flanges of the needle unit to fixedly mount the needle unit within the housing. The proximal end of the housing together with the at least two consecutive transverse walls or projections of each of the first housing portion and the second housing portion define a channel that is sized and shaped for receiving a syringe tip (e.g. a syringe tip having standard Luer dimensions) for engagement with the needle hub. The distal end of the housing comprises a first contact surface adapted to be placed on a skin of the subject and a second contact surface, wherein the first end of the needle shaft extends out of the second contact surface by a predefined distance for limiting a penetration depth of the needle shaft.

In another embodiment, the first housing portion and the second housing portion are configured for snap-fit engagement with one another to form the housing. As the skilled worker will appreciate, snap-fit engagement of the first and second housing portions is a very simple form of attachment that is fast and easily automated, which offers advantages over gluing (the limitations of which are discussed above) or other attachment methods, such as ultrasonic welding (which may not work for welding certain plastics together, and which would add complexity and cost to an automated assembly line). In another embodiment, the first housing portion and the second housing portion are of at least substantially similar or identical construction (which reduces tooling requirements and makes the device more economical to manufacture).

In another embodiment, the at least a portion of one or both of the pair of radially extending diametrically opposing flanges of the needle unit has a frictional engagement with opposing surfaces of the at least two consecutive transverse walls or projections of each of the first housing portion and the second housing portion when received in the gap therebetween.

In yet another embodiment, the gap formed by the at least two consecutive transverse walls or projections of each of the first housing portion and the second housing portion is configured to receive the at least a portion of one or both of the pair of radially extending diametrically opposing flanges of the needle unit in one of a first orientation and a second orientation of mounting of the needle unit, wherein: the predefined distance by which the first end of the needle shaft extends out of the second contact surface is a first predefined distance when the needle unit is mounted in the first orientation, and the predefined distance by which the first end of the needle shaft extends out of the second contact surface is a second predefined distance when the needle unit is mounted in the second orientation, wherein the first predefined distance is different from the second predefined distance.

In another embodiment, each of the first housing portion and the second housing portion further comprises a plurality of projections extending from the inner surface of a distal end thereof to form a needle guide configured to hold the needle shaft in place.

In another embodiment, the plurality of projections comprises at least two needle-stabilizing projections disposed on either side of the needle shaft and offset from one another along a longitudinal axis of the needle shaft, each of the at least two needle-stabilizing projections having a sloped surface abutting the needle shaft.

In yet another embodiment, the first contact surface is disposed along the perimeter of the distal end of the housing, and the second contact surface is substantially centrally disposed at the distal end of the housing. The second contact surface can be disposed at an end of a needle-stabilizing protrusion which can extend substantially centrally from the distal end of the housing.

In still yet another embodiment, the housing is generally cylindrical in shape.

In another embodiment, each of the first housing portion and the second housing portion is generally semi-cylindrical in shape.

In another embodiment, there is provided a method for assembling the above-defined needle adaptor, the method comprising: obtaining the first housing portion and the second housing portion; obtaining the needle unit; optionally, measuring a length of the needle shaft, and removing a preselected portion of a distal end of each of the first housing portion and the second housing portion based on the length of the needle shaft; mounting the needle unit in one of the first housing portion and the second housing portion by inserting the at least a portion of one or both of the pair of radially extending diametrically opposing flanges of the needle unit into the gap formed between the at least two consecutive transverse walls or projections; and engaging the first housing portion and the second housing portion with one another to form the housing.

In yet another embodiment, there is provided a method for assembling the above-defined needle adaptor, the method comprising: obtaining the first housing portion and the second housing portion; obtaining the needle unit; measuring a length of the needle shaft; determining whether the needle unit is to be mounted in the above-noted first orientation or the above-noted second orientation based on the length of the needle shaft; optionally, removing a preselected portion of a distal end of each of the first housing portion and the second housing portion based on the length of the needle shaft and based on whether the needle unit is to be mounted in the first orientation or the second orientation; mounting the needle unit in one of the first housing portion and the second housing portion in the first orientation or the second orientation by inserting the at least a portion of one or both of the pair of radially extending diametrically opposing flanges of the needle unit into the gap formed between the at least two consecutive transverse walls or projections; and engaging the first housing portion and the second housing portion with one another to form the housing.

In another embodiment of the above-described method for assembling the above-defined needle adaptor, removing the preselected portion of the distal end of each of the first housing portion and the second housing portion comprising cutting the preselected portion of the distal end of each of the first housing portion and the second housing portion, such as by laser cutting.

In still yet another embodiment of the above-described method for assembling the above-defined needle adaptor, the method is automated.

In another embodiment, there is provided an assembly for forming an injection device for administering a fluid to a subject, the assembly comprising a foot comprising a first contact surface adapted to be placed on a skin of the subject, the foot having a tubular shape for receiving a needle adaptor body, and a needle adaptor body. The needle adaptor body comprises a housing formed from a first housing portion and a second housing portion, the housing having a proximal end and a distal end; and a needle unit fixedly mounted within the housing. The needle unit comprises a needle shaft comprising a first end for penetrating the subject's skin and a second end connected to a needle hub, the needle hub comprising a distal end connected to the second end of the needle shaft and a proximal end comprising a pair of radially extending diametrically opposing flanges (e.g. typical needle hub tabs that would be found on commercially available needle units comprising a needle shaft and hub having a standard female Luer-Lok fitting). Each of the first housing portion and the second housing portion comprises at least two consecutive transverse walls or projections extending from an inner surface thereof, wherein the at least two consecutive transverse walls or projections form a gap therebetween for receiving at least a portion of one or both of the pair of radially extending diametrically opposing flanges of the needle unit to fixedly mount the needle unit within the housing. The proximal end of the housing together with the at least two consecutive transverse walls or projections of each of the first housing portion and the second housing portion define a channel that is sized and shaped for receiving a syringe tip (e.g. a syringe tip having standard Luer dimensions) for engagement with the needle hub. The distal end of the housing comprises a second contact surface, wherein the first end of the needle shaft extends out of the second contact surface by a predefined distance for limiting a penetration depth of the needle shaft. The needle adaptor body is movably mounted to the foot for allowing movement of the needle adaptor body from a first position to a second position, wherein when the needle adaptor body is in the first position, the needle shaft is in a retracted position such that the first end of the needle shaft does not extend beyond the first contact surface, and when the needle adaptor body is in the second position, the first end of the needle shaft extends beyond the first contact surface and out of the second contact surface by the predefined distance for limiting the penetration depth of the needle shaft. The assembly further comprises a friction means for inhibiting movement of the needle adaptor body relative to the foot when the needle adaptor body is in the first position, until a predefined static friction force is overcome, and for causing or allowing a sudden acceleration of the needle adaptor body towards the foot for increasing a speed of the needle shaft for increasing chance of penetration of the skin.

In another embodiment, the gap formed by the at least two consecutive transverse walls or projections of each of the first housing portion and the second housing portion is configured to receive the at least a portion of one or both of the pair of radially extending diametrically opposing flanges of the needle unit in one of a first orientation and a second orientation of mounting of the needle unit, wherein: the predefined distance by which the first end of the needle shaft extends out of the second contact surface is a first predefined distance when the needle unit is mounted in the first orientation, and the predefined distance by which the first end of the needle shaft extends out of the second contact surface is a second predefined distance when the needle unit is mounted in the second orientation, wherein the first predefined distance is different from the second predefined distance.

In another embodiment, the at least a portion of one or both of the pair of radially extending diametrically opposing flanges of the needle unit has a frictional engagement with opposing surfaces of the at least two consecutive transverse walls or projections of each of the first housing portion and the second housing portion when received in the gap therebetween.

In another embodiment, the first housing portion and the second housing portion are configured for snap-fit engagement with one another to form the housing. As noted above, snap-fit engagement of the first and second housing portions is a very simple form of attachment which offers advantages over gluing (the limitations of which are discussed above) or other attachment methods, such as ultrasonic welding. In another embodiment, the first housing portion and the second housing portion are of at least substantially similar or identical construction (which reduces tooling requirements and makes the device more economical to manufacture).

In yet another embodiment, each of the first housing portion and the second housing portion further comprises a plurality of projections extending from the inner surface of a distal end thereof to form a needle guide configured to hold the needle shaft in place.

In still yet another embodiment, the plurality of projections comprises at least two needle-stabilizing projections disposed on either side of the needle shaft and offset from one another along a longitudinal axis of the needle shaft, each of the at least two needle-stabilizing projections having a sloped surface abutting the needle shaft.

In another embodiment, the first contact surface is disposed along the perimeter of a distal end of the foot, and the second contact surface is substantially centrally disposed at the distal end of the housing. The second contact surface can be disposed at an end of a needle-stabilizing protrusion which can extend substantially centrally from the distal end of the housing.

In still yet another embodiment, the housing is generally cylindrical in shape. In another embodiment, each of the first housing portion and the second housing portion is generally semi-cylindrical in shape.

In another embodiment, the first friction means comprises at least two protrusions extending from an inner surface of a proximal end of the foot being in contact with at least two corresponding grooves located on an outer surface of the distal end of the housing of the needle adaptor body, wherein a radial dimension defined by the at least two protrusions before assembly of the needle adaptor body and the foot, is smaller than a radial dimension defined by the at least two corresponding grooves, the static friction being provided by radial clamping. However, it will be understood that the first friction means could equivalently comprise at least two protrusions extending from an outer surface of the body being in contact with at least two corresponding grooves located on an inner surface of the foot, wherein a radial dimension defined by the at least two protrusions before assembly of the body and the foot, is larger than a radial dimension defined by the grooves, the static friction being provided by radial clamping.

In yet another embodiment, the at least two corresponding grooves are configured to prevent disengagement of the foot from the needle adaptor body by limiting movement of the foot in an axial direction away from the needle adaptor body following engagement of the at least two protrusions extending from the inner surface of the proximal end of the foot with the at least two corresponding grooves.

In another embodiment, the at least two corresponding grooves are oriented generally parallel to a longitudinal axis of the housing.

In another embodiment, the assembly further comprises at least two deactivation grooves located on the outer surface of the distal end of the housing of the needle adaptor body, wherein each of the at least two deactivation grooves intersects one of the at least two corresponding grooves at an angle (e.g. about 25° to about 65°, e.g. about 45°) relative to the longitudinal axis of the housing, such that axial movement of the foot away from the needle adaptor body and rotation of the foot relative to the needle adaptor body engages the at least two protrusions with the at least two deactivation grooves, wherein each of the at least two deactivation grooves comprises an indentation complementary to a shape of each of the at least two protrusions to fixedly engage each of the at least two protrusions, such that the needle adaptor body is held in a fixed, deactivated position relative to the foot, wherein the first end of the needle shaft does not extend beyond the first contact surface when the needle adaptor body is in the fixed, deactivated position relative to the foot.

In yet another embodiment, the assembly further comprises a locking mechanism for providing a locked mode and an unlocked mode of the device, the locked mode being a mode of the assembly, wherein the needle adaptor body is prevented from moving axially towards the foot, even when an axial force larger than the predefined static friction is exerted on the needle adaptor body relative to the foot; the unlocked mode being a mode of the assembly wherein the needle adaptor body is allowed to move towards the foot, when an axial force larger than the predefined static friction is applied to the needle adaptor body relative to the foot. In another embodiment, the locking mechanism comprises a removable safety clip configured to engage with a portion of the outer surface of the housing to maintain the foot and needle adaptor body spaced apart from one another to prevent the needle adaptor body from moving axially towards the foot.

In another embodiment, a method for assembling the above-described assembly is provided, wherein the assembly optionally further comprises a locking mechanism comprising a removable safety clip configured to engage with a portion of the outer surface of the housing to maintain the foot and needle adaptor body spaced apart from one another to prevent the needle adaptor body from moving axially towards the foot, the method comprising: obtaining the foot; obtaining the first housing portion and the second housing portion forming the housing of the needle adaptor body; obtaining the needle unit; optionally, obtaining the removable safety clip; optionally, measuring a length of the needle shaft, and removing a preselected portion of a distal end of each of the first housing portion and the second housing portion based on the length of the needle shaft; mounting the needle unit in one of the first housing portion and the second housing portion by inserting the at least a portion of one or both of the pair of radially extending diametrically opposing flanges of the needle unit into the gap formed between the at least two consecutive transverse walls or projections; engaging the first housing portion and the second housing portion with one another to form the housing of the needle adaptor body; engaging the removable safety clip, if present, with the portion of the outer surface of the housing; and engaging the foot and the needle adaptor body.

In yet another embodiment, a method for assembling the above-described assembly is provided wherein the assembly optionally further comprises a locking mechanism comprising a removable safety clip configured to engage with a portion of the outer surface of the housing to maintain the foot and needle adaptor body spaced apart from one another to prevent the needle adaptor body from moving axially towards the foot, the method comprising: obtaining the foot; obtaining the first housing portion and the second housing portion forming the housing of the needle adaptor body; obtaining the needle unit; optionally, obtaining the removable safety clip; measuring a length of the needle shaft; determining whether the needle unit is to be mounted in the first orientation or the second orientation based on the length of the needle shaft; optionally, removing a preselected portion of a distal end of each of the first housing portion and the second housing portion based on the length of the needle shaft and based on whether the needle unit is to be mounted in the first orientation or the second orientation; mounting the needle unit in one of the first housing portion and the second housing portion in the first orientation or the second orientation by inserting the at least a portion of one or both of the pair of radially extending diametrically opposing flanges of the needle unit into the gap formed between the at least two consecutive transverse walls or projections; and engaging the first housing portion and the second housing portion with one another to form the housing of the needle adaptor body; engaging the removable safety clip, if present, with the portion of the outer surface of the housing; and engaging the foot and the needle adaptor body.

In another embodiment of the above-described method, engaging the foot and the needle adaptor body comprises engaging the at least two protrusions extending from the inner surface of the proximal end of the foot with the at least two corresponding grooves located on the outer surface of the distal end of the housing of the needle adaptor body.

In another embodiment of the above-described method for assembling the above-defined assembly, removing the preselected portion of the distal end of each of the first housing portion and the second housing portion comprising cutting the preselected portion of the distal end of each of the first housing portion and the second housing portion, such as by laser cutting.

In still yet another embodiment of the above-described method for assembling the above-defined assembly, the method is automated.

In another embodiment, there is provided a method of administering a fluid to a subject via injection, the method comprising: (a) obtaining the above-described needle adaptor; (b) obtaining a syringe or other dosing device, wherein the other dosing device comprises a dispensing tip that is similar in size and shape to a syringe tip (the channel being sized and shaped for receiving the tip of the syringe/other dosing device for engagement with the needle hub), wherein the syringe or the other dosing device is loaded with the fluid to be administered to the subject; (c) inserting the tip of the syringe or the other dosing device into the channel disposed at the proximal end of the housing so as to engage the tip with the needle hub; (d) engaging the first contact surface with the skin of the subject; (e) pushing the housing against the skin to allow the first end of the needle shaft to penetrate the skin; (f) expelling the fluid from the syringe or the other dosing device through the needle shaft into the subject; and (g) optionally, engaging the needle adaptor with a safety holder, wherein the safety holder has an open end for receiving at least the distal end of the needle adaptor housing and a closed end, the closed end comprising opposed wings for stabilizing the safety holder on a horizontal surface.

In another embodiment, there is provided a method of administering a fluid to a subject via injection, the method comprising: (a) obtaining the above-described assembly, wherein the needle adaptor body is in the first position; (b) obtaining a syringe or other dosing device, wherein the other dosing device comprises a dispensing tip that is similar in size and shape to a syringe tip (the channel being sized and shaped for receiving the tip of the syringe/other dosing device for engagement with the needle hub), wherein the syringe or the other dosing device is loaded with the fluid to be administered to the subject; (c) inserting the tip of the syringe or the other dosing device into the channel disposed at the proximal end of the needle adaptor housing so as to engage the tip with the needle hub; (d) engaging the first contact surface of the foot with the skin of the subject; (e) pushing the housing of the needle adaptor body towards the foot to move the needle adaptor body from the first position to the second position, thus causing the first end of the needle shaft to penetrate the skin; and (f) expelling the fluid from the syringe or the other dosing device through the needle shaft into the subject.

In yet another embodiment wherein the above-described assembly comprises a needle adaptor housing with deactivation grooves, there is provided a method of administering a fluid to a subject via injection, the method comprising: (a) obtaining the assembly, wherein the needle adaptor body is in the first position; (b) obtaining a syringe or other dosing device, wherein the other dosing device comprises a dispensing tip that is similar in size and shape to a syringe tip (the channel being sized and shaped for receiving the tip of the syringe/other dosing device for engagement with the needle hub), wherein the syringe or the other dosing device is loaded with the fluid to be administered to the subject; (c) inserting the tip of the syringe or the other dosing device into the channel disposed at the proximal end of the needle adaptor housing so as to engage the tip with the needle hub; (d) engaging the first contact surface of the foot with the skin of the subject; (e) pushing the housing of the needle adaptor body towards the foot in an axial direction to move the needle adaptor body from the first position to the second position, thus causing the first end of the needle shaft to penetrate the skin; (f) expelling the fluid from the syringe or the other dosing device through the needle shaft into the subject; and (g) pulling the housing of the needle adaptor body away from the foot in an axial direction and rotating the foot relative to the needle adaptor body to engage the at least two protrusions with the at least two deactivation grooves and to fixedly engage each of the at least two protrusions in the indentation in each of the at least two deactivation grooves, such that the needle adaptor body is held in the fixed, deactivated position relative to the foot.

In still yet another embodiment wherein the above-described assembly comprises a needle adaptor housing with deactivation grooves as well as a locking mechanism comprising a removable safety clip, there is provided a method of administering a fluid to a subject via injection, the method comprising: (a) obtaining the above-described assembly, wherein the needle adaptor body is in the first position; (b) obtaining a syringe or other dosing device, wherein the other dosing device comprises a dispensing tip that is similar in size and shape to a syringe tip (the channel being sized and shaped for receiving the tip of the syringe/other dosing device for engagement with the needle hub), wherein the syringe or the other dosing device is loaded with the fluid to be administered to the subject; (c) inserting the tip of the syringe or the other dosing device into the channel disposed at the proximal end of the needle adaptor housing so as to engage the tip with the needle hub; (d) engaging the first contact surface of the foot with the skin of the subject; (e) pushing the housing of the needle adaptor body towards the foot in an axial direction to move the needle adaptor body from the first position to the second position, thus causing the first end of the needle shaft to penetrate the skin; (f) expelling the fluid from the syringe or the other dosing device through the needle shaft into the subject; and (g) pulling the housing of the needle adaptor body away from the foot in an axial direction and rotating the foot relative to the needle adaptor body to engage the at least two protrusions with the at least two deactivation grooves and to fixedly engage each of the at least two protrusions in the indentation in each of the at least two deactivation grooves, such that the needle adaptor body is held in the fixed, deactivated position relative to the foot; the method further comprising removing the safety clip from the outer surface of the housing after step (c) and prior to step (d), or after step (d) and prior to step (e).

Other dosing devices that could be used in place of a syringe with the needle adaptor and assembly of the present application could include multi-chamber pre-filled containers (e.g. dual chambers for lyophilized substances and diluents), with a means for mixing the components of the chambers and means for expelling same from the dosing device (e.g. by way of a plunger, etc.).

In another embodiment, there is provided a kit comprising: the above-described needle adaptor for forming an injection device for administering a fluid to a subject; a syringe or other dosing device, wherein the other dosing device comprises a dispensing tip that is similar in size and shape to a syringe tip, wherein the syringe or the other dosing device is optionally loaded with the fluid to be administered to the subject; optionally, a vial containing the fluid to be administered to the subject; optionally, a removable needle unit or other means for extracting the fluid from the optional vial into the syringe or the other dosing device, the removable needle unit being removable for allowing the tip of the syringe or the other dosing device to be inserted into the channel of the housing; optionally, a safety holder, wherein the safety holder has an open end for receiving at least the distal end of the needle adaptor housing and a closed end, the closed end comprising opposed wings for stabilizing the safety holder on a horizontal surface; and optionally, instructions for use.

In another embodiment, there is provided a kit comprising: the above-described assembly for forming an injection device for administering a fluid to a subject; a syringe or other dosing device, wherein the other dosing device comprises a dispensing tip that is similar in size and shape to a syringe tip, wherein the syringe or the other dosing device is optionally loaded with the fluid to be administered to the subject; optionally, a vial containing the fluid to be administered to the subject; optionally, a removable needle unit or other means for extracting the fluid from the optional vial into the syringe or the other dosing device, the removable needle unit being removable for allowing the tip of the syringe or the other dosing device to be inserted into the channel of the housing; and optionally, instructions for use.

In yet another embodiment, there is provided an injection device comprising: the above-described needle adaptor; and a syringe or other dosing device, wherein the other dosing device comprises a dispensing tip that is similar in size and shape to a syringe tip, wherein the syringe or the other dosing device is optionally loaded with the fluid to be administered to the subject.

In still yet another embodiment, there is provided an injection device comprising: the above-described assembly; and a syringe or other dosing device, wherein the other dosing device comprises a dispensing tip that is similar in size and shape to a syringe tip, wherein the syringe or the other dosing device is optionally loaded with the fluid to be administered to the subject.

The various parts of the needle adaptor, as well as the assembly for forming an injection device for administering a fluid to a subject, can be formed from plastic materials, in particular Medical Grade plastic (e.g. Cyclic Olefin Copolymer (COC)) and can be manufactured by a number of different methods, such as precision casting, additive manufacturing, 3D-printing, and injection moulding. In one embodiment, the parts are manufactured using injection moulding. The tolerances of such processes can be precisely controlled, for example in the order of 0.01 mm or 0.02 mm or 0.03 mm, which allows for accurate construction of the devices including accurate implementation of the friction forces described in further detail below.

It will be apparent to the skilled worker that the present needle adaptor, as well as the assembly for forming an injection device for administering a fluid to a subject, can be used to administer various drugs or vaccines. These devices are especially suitable for providing injections at a very precise angle and/or penetration depth, such as for example for ID-injections with the needle being oriented nearly perpendicular to the skin and being inserted typically to a very precise and predefined depth of for example about 1.0 mm with a tolerance of +/−0.10 mm or +/−0.05 mm, or even smaller, but other specific angles can also be used. However, it will be understood that the present invention is not limited to ID-injections, and can also be used for IV, SC, or IM injections, although in these cases the needle would typically have a much larger length, for example at least 5 mm or at least 10 mm. As would be appreciated by the skilled worker, the angle and/or penetration depth and/or the positioning of the device may be chosen differently for such types of injections.

In one embodiment, the needle adaptor described herein allows for the fluid to be administered by a single hand, and thus such devices are suitable for self-administration. For example, in respect of the needle adaptor, a syringe can be loaded with an active agent-containing fluid, the syringe having a plunger for dispensing same. The user can then insert the tip of the syringe into the channel disposed at the proximal end of the housing. The first contact surface of the needle adaptor can then be placed on the skin and the needle adaptor can be pressed into the skin to insert the first end of the needle shaft into the skin. Finally, force can then be applied to the plunger of the syringe (e.g. with the forefinger or index finger) to deliver the fluid through the needle shaft into the body of the user.

In another embodiment, the needle adaptor can be used to deliver multiple doses of a liquid. In another embodiment, the needle adaptor can be coupled to a dose-metering device that is compatible with a syringe (wherein the tip of the syringe enters the channel of the device and the dose-metering device controls the amount of fluid being delivered in a single dose). In another embodiment, the needle adaptor can be coupled to another dosing device that has a dispensing tip that is similar in size and shape to a syringe tip, such as a syringe tip having standard Luer dimensions.

In another embodiment, the assembly described herein allows for the fluid to be administered by a single hand, and thus such devices are suitable for self-administration. For example, a syringe can be loaded with an active agent-containing fluid, the syringe having a plunger for dispensing same. The user can then insert the tip of the syringe into the channel disposed at the proximal end of the housing of the needle adaptor body. The steps of administration may comprise: 1) holding the assembly with one hand (e.g. between the thumb and the middle finger), 2) gently placing the assembly on the skin, 3) pushing the needle adaptor body towards the foot until the friction force is overcome, thereby inserting the first end of the needle shaft in the skin (with almost 100% probability of penetration, and with a highly accurate predefined penetration depth), and 4) applying force to the plunger of the syringe (e.g. with the forefinger or index finger) to deliver the fluid through the needle shaft to the subject. In other embodiments, the steps for administration can include disengagement of a locking mechanism, such as a removable safety clip, to activate the device prior to pushing the needle adaptor body toward the foot to insert the first end of the needle shaft into the skin, as described above and in further detail below. In another embodiment, the steps for administration can include placing the needle adaptor body in a fixed, deactivated position relative to the foot following administration of the fluid to the subject. The assembly described herein is particularly suitable for single use.

In another embodiment, the needle adaptor body can be coupled to another dosing device that has a dispensing tip that is similar in size and shape to a syringe tip, such as a syringe tip having standard Luer dimensions. It will be understood that the channel is sized and shaped for receiving the tip of the syringe/other dosing device for engagement with the needle hub.

It will be further appreciated that the present needle adaptor, as well as the assembly for forming an injection device for administering a fluid to a subject require only minimal skill and experience to correctly administer a fluid, in contrast to, for example, the Mantoux technique of administering ID injections. In addition, the risk of non-penetration or incomplete penetration (to the predefined penetration depth) of the needle shaft in the skin, is drastically reduced or almost completely eliminated, as is the risk of inserting the needle shaft too deeply. Thus, with the present needle adaptor and assembly, it is almost guaranteed that the skin will be penetrated, and that the needle tip will be located at a predefined depth. This may help to reduce the pain experienced by the subject, and/or to improve the therapeutic effect of the active agent that is being administered.

In respect of the above-described assembly, no spring is required for inserting the needle shaft (and as such no internal or external mechanism for compressing, holding, and releasing such spring), but instead, with the assembly of the present application, a force/pressure/potential energy and/or kinetic energy is built up in/provided by the hand and/or forearm and/or fingers of the person holding the assembly, yet the device contains a mechanism (by means of the static friction force) that enables or disables this (external) force to have an effect. A spring may be used in an injection device using this assembly, for example to actuate a plunger, but this is unrelated to the insertion of the needle shaft in the skin.

The friction means, which sets or defines the force/pressure/potential energy to be build-up before the needle starts to move, can be well defined in a passive manner, e.g. by a clamping force between portions of the needle adaptor body (also referred to herein as “body”) and the foot (described in further detail below). This will cause the needle to suddenly accelerate when the static friction force is overcome, so that the needle will penetrate the skin with a relatively high speed (e.g. between 2 m/s and 15 m/s, or any other suitable speed). The predefined static friction force can be a value in the range from about 1.0 to about 20.0 Newton, or from about 1.5 to about 15 Newton, or from about 2.0 to about 10 Newton, or from about 5.0 to about 7.5 Newton; preferably the static friction force is at least about 2.0 Newton. The optimum penetration speed, and thus the optimum friction may be chosen differently for different needle units (e.g. different diameter, different length, different angles, etc.), and different customized assemblies (e.g. having different surface characteristics of the above-noted grooves and/or of the protrusions) can be made having different needle units.

In one embodiment, an angle between a longitudinal axis of the needle shaft and a tangential plane defined by the first contact surface is a value in the range of, for example, from about 5° to about 175°, from about 10° to about 170°, from about 60° to about 120°, for example from about 80° to about 100°, e.g. about 90°. Thus, the present needle adaptor and assembly allow for ID injections at a predefined angle, which angle is different from the Mantoux-technique, which administers ID drugs under an angle of about 5° to about 15° and which is known to be painful to the patient. It is thought that inserting the needle under an angle close to 90° will be significantly less painful, and may also allow the injected fluid to spread better between the cells.

In one embodiment, the predefined distance by which the at least one needle shaft extends out of the second contact surface is a distance in the range of 0.25 to 12.0 mm, or from 0.25 to 5.00 mm, or from 0.25 to 2.00 mm. A distance from 5.0 mm to 12.0 mm, for example from 10 mm to 120 mm may be especially suitable for IM injections. A distance from 0.25 mm to 8.00 mm, for example from 1.00 mm to 5.00 mm may be especially suitable for SC injections. A distance from 0.25 mm to 3.00 mm may be especially suitable for ID injections.

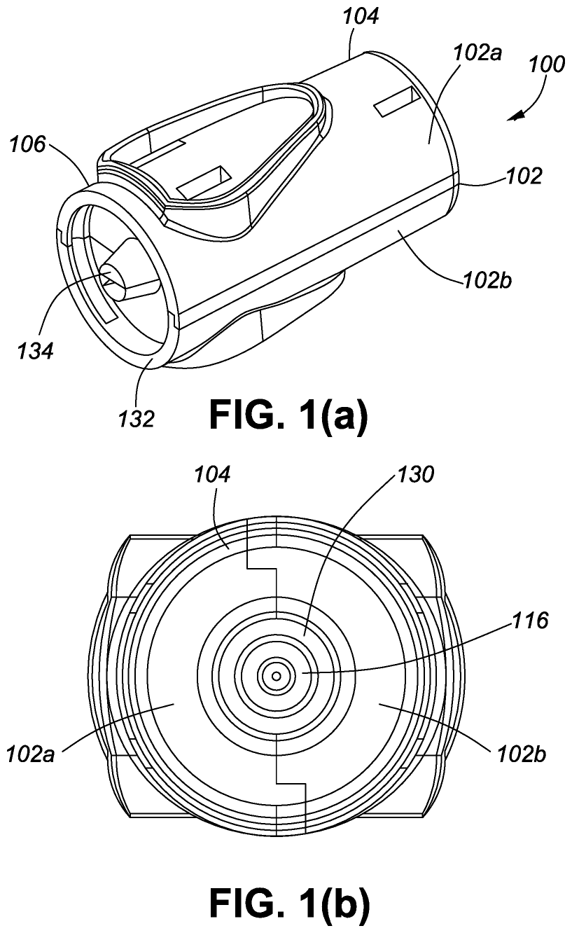

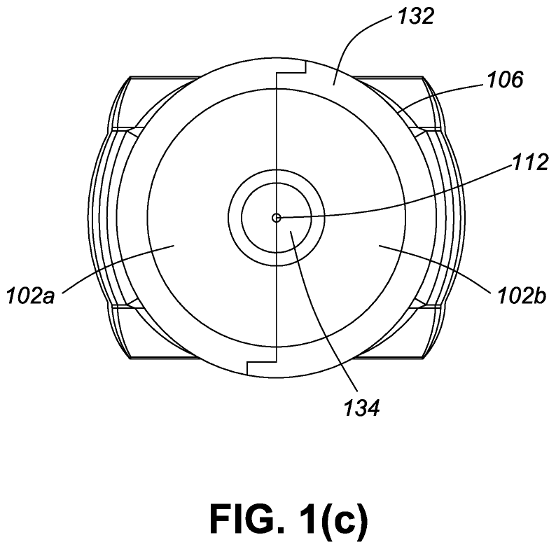

FIG. 1(a) illustrates an exemplary needle adaptor 100 according to an embodiment of the present application, in perspective view. The needle adaptor 100 includes a housing 102 formed from a first housing portion 102a and a second housing portion 102b, the housing having a proximal end 104 and a distal end 106. FIG. 1(b) illustrates a top view of the needle adaptor 100 shown in FIG. 1(a), showing the proximal end 104 of the housing 102 of the needle adaptor 100 of FIG. 1(a). FIG. 1(c) illustrates a bottom view of the needle adaptor shown in FIG. 1(a). In the present embodiment, the first housing portion 102a and the second housing portion 102b are of identical construction and can be formed using injection moulding of Medical Grade plastic (e.g. COC), which results in a very economical production. In the embodiment shown, the housing 102 is generally cylindrical in shape, and each of the first housing portion 102a and the second housing portion 102b is generally semi-cylindrical in shape.

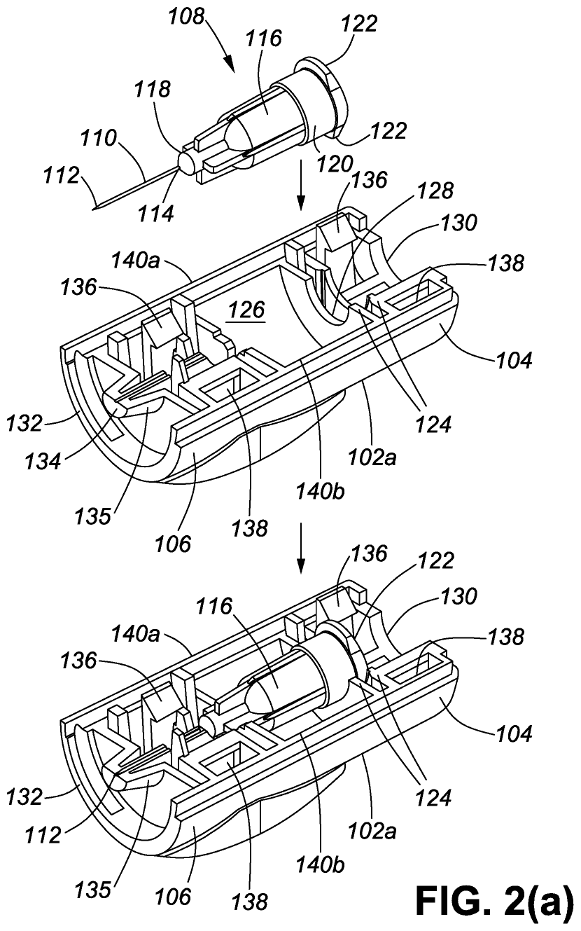

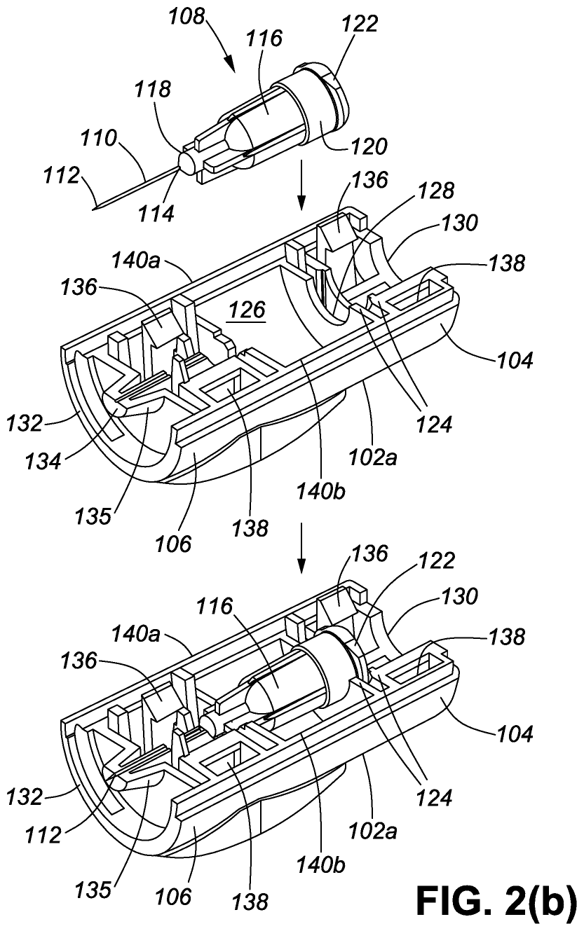

A needle unit 108 is fixedly mounted within the housing 102. FIGS. 2(a) and 2(b) illustrate the first housing portion 102a and the manner in which the needle unit 108 can be engaged therewith, namely in a first orientation as shown in FIG. 2(a), and a second orientation as shown in FIG. 2(b). Following engagement of the needle unit 108 with the first housing portion 102a, the second housing portion 102b is then engaged with the first housing portion 102a to form the needle adaptor 100. As the skilled worker will appreciate, it is equally possible for the needle unit to engage with the second housing portion 102b in the same manner as shown for the first housing portion 102a in FIGS. 2(a) and 2(b), given the first housing portion 102a and the second housing portion 102b are of identical construction.

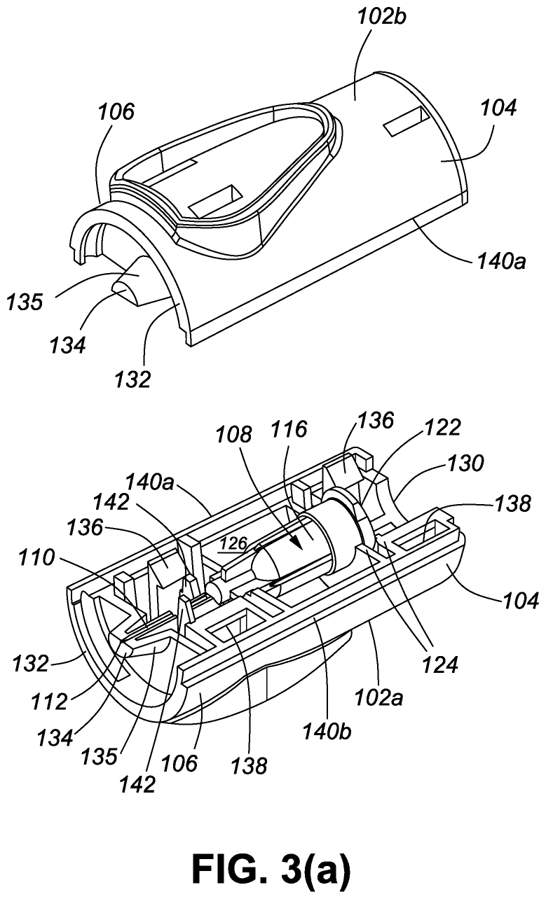

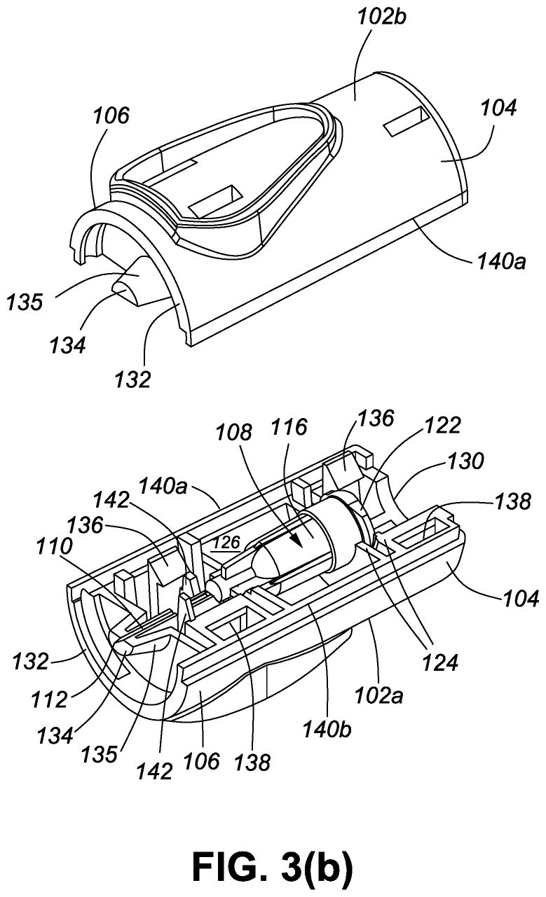

FIG. 3(a) shows the component parts of the needle adaptor 100 with the needle unit 108 engaged with the first housing portion 102a in the first orientation, together with the second housing portion 102b which is configured to engage with the first housing portion 102a to form the housing 102. FIG. 3(b) shows the component parts of the needle adaptor 100 with the needle unit 108 engaged with the first housing portion 102a in the second orientation, together with the second housing portion 102b which is configured to engage with the first housing portion 102a to form the housing 102.

As can be best seen from FIGS. 2(a) and 2(b) and FIGS. 3(a) and 3(b), the needle unit 108 comprises: a needle shaft 110 comprising a first end 112 for penetrating the subject's skin and a second end 114 connected to a needle hub 116. The needle hub 116 comprises a distal end 118 connected to the second end of the needle shaft 114 and a proximal end 120 comprising a pair of radially extending diametrically opposing flanges 122. In the embodiment shown, the needle hub 116 has typical needle hub tabs that would be found on commercially available needle units comprising a needle shaft and hub having a standard female Luer-Lok fitting. Each of the first housing portion 102a and the second housing portion 102b comprises at least two consecutive transverse walls or projections 124 extending from an inner surface 126 thereof, wherein the at least two consecutive transverse walls or projections 124 form a gap 128 therebetween for receiving at least a portion of one or both of the pair of radially extending diametrically opposing flanges 122 of the needle unit 108 to fixedly mount the needle unit 108 within the housing 102. The proximal end 104 of the housing 102 together with the at least two consecutive transverse walls or projections 124 of each of the first housing portion 102a and the second housing portion 102b define a channel 130 for receiving a syringe tip for engagement with the needle hub 116. The distal end 106 of the housing 102 comprises a first contact surface 132 adapted to be placed on a skin of the subject and a second contact surface 134, wherein the first end of the needle shaft extends out of the second contact surface by a predefined distance “d1” for limiting a penetration depth of the needle shaft 110. In the embodiment shown, the first contact surface 132 is disposed along the perimeter of the distal end 106 of the housing 102, and the second contact surface 134 is substantially centrally disposed at the distal end 106 of the housing 102. Specifically, the second contact surface is disposed at an end of a needle-stabilizing protrusion 135 which extends substantially centrally from the distal end of the housing.

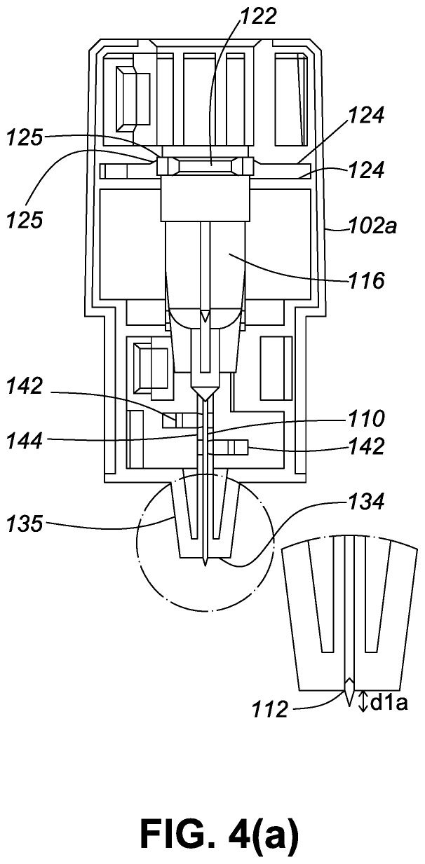

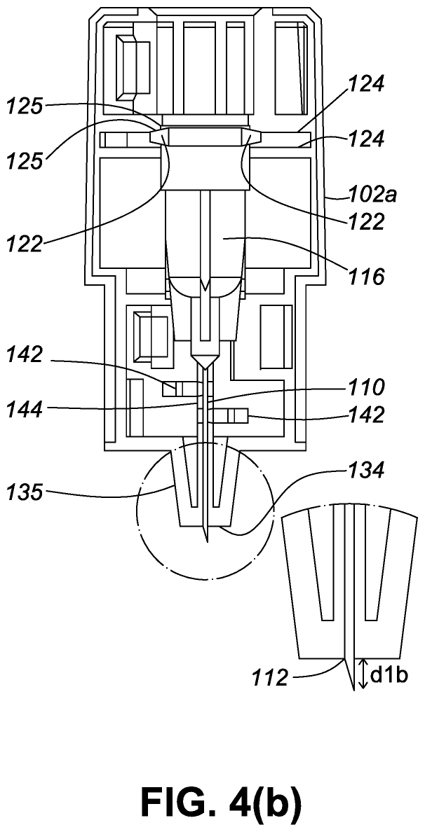

FIGS. 4(a) and 4(b) illustrate a simplified first housing portion 102a of the needle adaptor 100 wherein the distal end of the housing 106 lacks the first contact surface 132, in order to better illustrate how varying the placement of the needle unit 108 within the first housing portion 102a impacts the predefined distance d1 that the first end of the needle shaft 112 extends out of the second contact surface 134. As noted above, as a result of current state-of-the-art manufacturing processes, it is known that final needle lengths will be subject to production tolerances of e.g. 0.05 mm, or e.g. 2 mm as defined in specific ISO standards. The needle adaptor described herein allows for control over penetration depth regardless of intended needle length and tolerance deviations. The needle adaptor of the present application allows for the use of needle units having longer needle shafts, such as (pre-glued) commercially available needle units comprising a needle shaft and hub having a standard female Luer-Lok fitting, e.g. 26-34 G and 12 mm length. Such needle units can have long needle shafts with broad tolerances, while the needle adaptor of the present application can accurately control penetration depth irrespective of same. The needle adaptor of the present application can therefore account for and offset manufacturer variability in needle shafts.

As can be seen in FIGS. 2(a) and 2(b), 3(a) and 3(b), and 4(a) and 4(b), the gap 128 formed by the at least two consecutive transverse walls or projections 124 of the first housing portion 102a is configured to receive the at least a portion of one or both of the pair of radially extending diametrically opposing flanges 122 of the needle unit 108 in one of a first orientation (FIG. 4(a)) and a second orientation (FIG. 4(b)) of mounting of the needle unit. As best seen in FIGS. 4(a) and 4(b), the first and second orientation of mounting of the needle unit can differ from one another by about a 90 degree rotation. Stepped stops or shoulders 125 are present in at least one of the at least two consecutive transverse walls or projections 124 of the first housing portion 102a, which engage with the distal end 118 of the needle hub 116 and facilitate fixedly mounting the needle unit 108 in the first orientation or the second orientation. The flanges 122 of the needle unit 108 have a frictional engagement with opposing surfaces of the at least two consecutive transverse walls or projections 124 of each of the first housing portion 102a and the second housing portion 102b when received in the gap 128 therebetween, thus avoiding the need to use less desirable means for fixedly engaging the needle unit within the housing, such as gluing, overmoulding, etc. as discussed above.

As best shown in FIG. 4(a), the predefined distance d1 by which the first end 112 of the needle shaft 110 extends out of the second contact surface 134 is a first predefined distance d1a when the needle unit 108 is mounted in the first orientation (i.e. when the distal end 118 of the needle hub 116 is oriented with the pair of radially extending diametrically opposing flanges 122 extending directly into, and out of, the plane of the paper in FIG. 4(a)—this is also referred to herein as the flanges being disposed in a “vertical” position, or Position V). As best shown in FIG. 4(b), the predefined distance d1 by which the first end 112 of the needle shaft 110 extends out of the second contact surface 134 is a second predefined distance d1b when the needle unit 108 is mounted in the second orientation (i.e. when the distal end 118 of the needle hub 116 is oriented with the pair of radially extending diametrically opposing flanges 122 extending from side-to-side as shown in FIG. 4(b)—this is also referred to herein as the flanges being disposed in a “horizontal” position, or Position H). As illustrated in FIGS. 4(a) and 4(b), the first predefined distance d1a is different from the second predefined distance d1 b.

Again, while the above-noted description is with reference to the first housing portion 102a, it will be understood that the first housing portion 102a is interchangeable with the second housing portion 102b, given they are of identical construction.

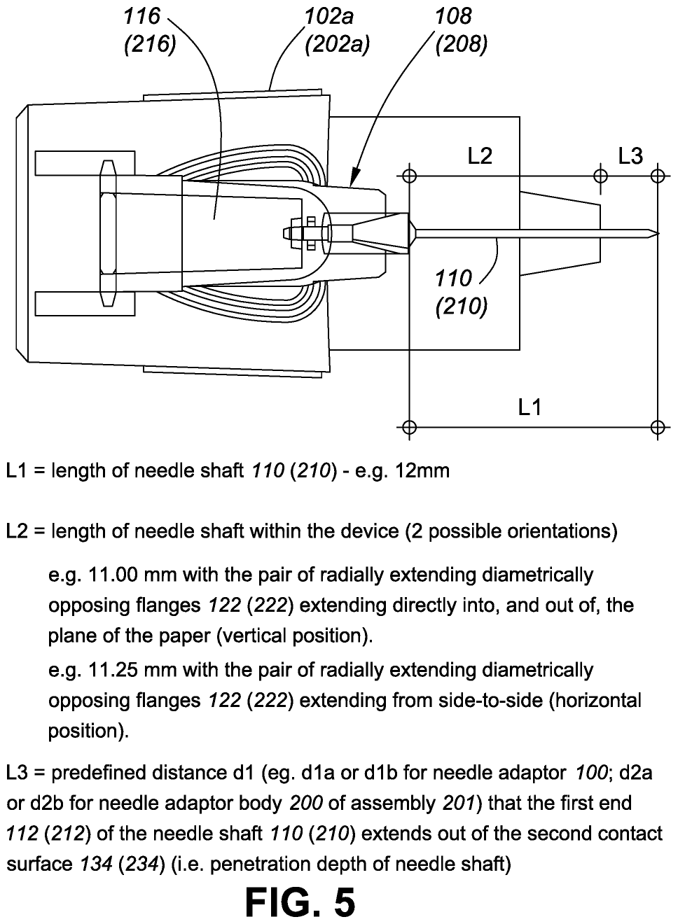

FIG. 5 represents a further simplified view of the simplified first housing portion 102a shown in FIGS. 4(a) and 4(b) and also represents a simplified first housing position 202a of the needle adaptor body 200 of the assembly 201 discussed in further detail below. In the labels for FIG. 5 and in the following description, references to components/elements of the needle adaptor body 200 of the assembly 201 discussed below will be provided in brackets. The needle unit 108 (208) is engaged with the first housing portion 102a (202a). FIG. 5 illustrates the impact of the orientation of the needle unit 108 (208) on the predefined distance d1 (d2) that the first end 112 (212) of the needle shaft 110 (210) extends out of the second contact surface 134 (234). Table 1 illustrates this in further detail:

TABLE 1

| L1 = | L2 = | L3 = Predefined | | Delta |
|---|---|---|---|---|
| Actual | Length of | Distance d1 (d2) | Orientation | Relative |
| Length of | Needle Shaft | i.e. penetration | of Flanges | to Desired |
| Needle | Within | depth of needle | 122 | d1 (d2) of |
| Shaft | Device | shaft | (222) | 0.85 mm |
| 11.750 | 11.000 | 0.750 | Position H | −0.100 |
| 11.800 | 11.000 | 0.800 | Position H | −0.050 |
| 11.850 | 11.000 | 0.850 | Position H | 0.000 |
| 11.900 | 11.000 | 0.900 | Position H | 0.050 |
| 11.950 | 11.000 | 0.950 | Position H | 0.100 |
| 12.000 | 11.250 | 0.750 | Position V | −0.100 |
| 12.050 | 11.250 | 0.800 | Position V | −0.050 |
| 12.100 | 11.250 | 0.850 | Position V | 0.000 |
| 12.150 | 11.250 | 0.900 | Position V | 0.050 |
| 12.200 | 11.250 | 0.950 | Position V | 0.100 |

In FIG. 5, L1 indicates the length of the needle shaft 110 (210), e.g. 12 mm. L2 indicates the length of the needle shaft within the device (2 possible orientations), e.g. 11.00 mm with the pair of radially extending diametrically opposing flanges 122 (222) extending directly into, and out of, the plane of the paper (vertical position), and e.g. 11.25 mm with the pair of radially extending diametrically opposing flanges 122 (222) extending from side-to-side (horizontal position). L3 indicates the predefined distance d1 (e.g. d1a or d1b for needle adaptor 100; d2a or d2b for needle adaptor body 200 of assembly 201) that the first end 112 (212) of the needle shaft 110 (210) extends out of the second contact surface 134 (234) (i.e. penetration depth of needle shaft).

With reference to Table 1, in the case of a 31 G needle, an exemplary desired length L3 (predefined distance d1 (d2)) is 0.85 mm. It is further desired to have this value be within a specific tolerance of e.g. +1-0.10 mm (so, a tolerance width of 0.20 mm, ranging L3 from 0.75 to 0.95 mm). From the experience of the inventors, it is known that a standard 31 G needle of e.g. L1 12 mm has a manufacturing tolerance that can significantly exceed the desired specific tolerance of e.g. +1-0.10 mm. As such, in the absence of a means in the present needle adaptor to account for manufacturer variability in needle shafts, the ability to use commercially available needle units would be severely hampered. However, by having the ability to mount the needle unit 108 (208) in two different orientations within the housing 102 (202) to adjust the predefined distance d1 (d2) that the first end 112 (212) of the needle shaft 110 (210) extends from the second contact surface 134 (234), it is possible to virtually double the tolerance width to e.g. 0.45 mm, as illustrated in FIG. 5 and Table 1. For instance, if the 2 L2 positions have a difference in distance of e.g. 0.25 mm (11.00 versus 11.25 mm), it is possible to operate within the specific tolerance of e.g. +/−0.10 mm for needle shaft lengths ranging from 11.75 to 12.20 mm (which in the inventors' experience is more in line with reality). As such, the needle adaptor and assembly (discussed below) of the present application have a very significant benefit in that they can account for and offset manufacturer variability in needle shafts.

As can be seen from FIGS. 2(a) and (b), and 3(a) and (b), the first housing portion 102a and the second housing portion 102b are configured for snap-fit engagement with one another to form the housing 102. This is accomplished by way of fasteners (i.e. snaps) 136 which project from the inner surface of each of the first housing portion 102a and the second housing portion 102b, which are configured to engage with complementary slots 138 formed within an inner portion of the housing of each of the first housing portion 102a and the second housing portion 102b in a snap-fit engagement. Each of the first housing portion 102a and the second housing portion 102b also has complementary ribs 140a and 140b which further assist engagement of the first housing portion 102a and the second housing portion 102b to form the housing 102. As such, the snap-fit engagement of the first housing portion 102a and the second housing portion 102b to form the housing 102 is a simple and straightforward means for fixedly joining these two components of the housing 102 together, which does not require the use of glue or other means for joining these components.

As can be seen in particular from FIGS. 3(a) and (b) and FIGS. 4(a) and (b) (also present in FIGS. 2(a) and 2(b), although not specifically labelled for ease of reading the remainder of the figure labels), each of the first housing portion 102a and the second housing portion 102b further comprises a plurality of projections 142 extending from the inner surface 126 of a distal end thereof to form a needle guide 144 configured to hold the needle shaft 110 in place. In the embodiment shown, the plurality of projections comprises at least two needle-stabilizing projections 142 disposed on either side of the needle shaft 110 and offset from one another along a longitudinal axis of the needle shaft 110, each of the at least two needle-stabilizing projections 142 having a sloped surface abutting the needle shaft. These features of the needle adaptor stabilize the needle shaft and hold it in a fixed position upon assembly of the first housing portion 102a and the second housing portion 102b to form the housing 102.

In the embodiments shown in FIGS. 1(a)-(c), 2(a) and (b), etc. the angle between a longitudinal axis of the needle shaft 110 and a tangential plane defined by the first contact surface 132 is about 90°. It will be understood by the skilled worker that this angle can be varied (e.g. to be in the range of, for example, from about 5° to about 175°, from about 10° to about 170°, from about 60° to about 120°, for example from about 80° to about 100°) by adjusting the angle by which the at least two consecutive transverse walls or projections 124 extend from the inner surface 126 of the first/second housing portions (102a/102b) along with the positioning of other supporting features (e.g. needle guide 144).

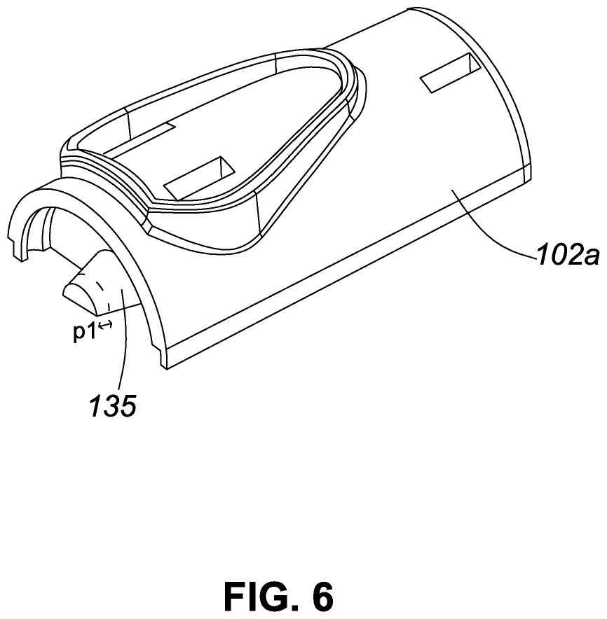

As noted above, the features of the needle adaptor 100 can account for and offset manufacturer variability in needle shafts by having the ability to mount the needle unit 108 in two different orientations within the housing 102. If it is necessary to further adjust the predefined distance d1 that the first end 112 of the needle shaft 110 extends from the second contact surface 134, this can be done during assembly of the needle adaptor. A method for assembling the needle adaptor can therefore comprise: obtaining the first housing portion 102a and the second housing portion 102b; obtaining the needle unit 108; measuring a length of the needle shaft 110; determining whether the needle unit 108 is to be mounted in the first orientation or the second orientation based on the length of the needle shaft 110; optionally, removing a preselected portion p1 of a distal end of each of the first housing portion 102a and the second housing portion 102b based on the length of the needle shaft 110 and based on whether the needle unit 108 is to be mounted in the first orientation or the second orientation. The needle unit can then be mounted in one of the first housing portion 102a and the second housing portion 102b in the first orientation or the second orientation by inserting the at least a portion of one or both of the pair of radially extending diametrically opposing flanges 122 of the needle unit 108 into the gap 128 formed between the at least two consecutive transverse walls or projections 124; and engaging the first housing portion 102a and the second housing portion 102b with one another to form the housing 102. FIG. 6 illustrates a perspective view of the first housing portion 102a, showing how a preselected portion p1 of a distal end of the first housing portion 102a can be removed during assembly of the needle adaptor 100 to further account for manufacturer variability in needle shaft lengths (the preselected portion p1 is shown in exaggerated detail, for ease of viewing). It will be understood that the same preselected portion p1 of a distal end of the second housing portion 102b would then also be removed during assembly of the needle adaptor 100.

Removing the preselected portion p1 of the distal end of each of the first housing portion 102a and the second housing portion 102b can comprise cutting the preselected portion p1 of the distal end of each of the first housing portion 102a and the second housing portion 102b, such as by laser cutting. The assembly process can further be automated such that a vision/imaging system (“Machine Vision”) on an automated assembly line (e.g. based on CCD cameras) can determine the length of the needle shaft (e.g. within 0.005 mm accuracy), orientation of the needle unit, and whether removal of a preselected portion of the distal end of each of the first housing portion and the second housing portion is required.

A method of administering a fluid to a subject via injection using the above-described needle adaptor can comprise: (a) obtaining the needle adaptor; (b) obtaining a syringe or other dosing device, wherein the other dosing device comprises a dispensing tip that is similar in size and shape to a syringe tip, wherein the syringe or the other dosing device is loaded with the fluid to be administered to the subject; (c) inserting the tip of the syringe or the other dosing device into the channel disposed at the proximal end of the housing so as to engage the tip with the needle hub; (d) engaging the first contact surface with the skin of the subject; (e) pushing the housing against the skin to allow the first end of the needle shaft to penetrate the skin; and (f) expelling the fluid from the syringe or the other dosing device through the needle shaft into the subject.

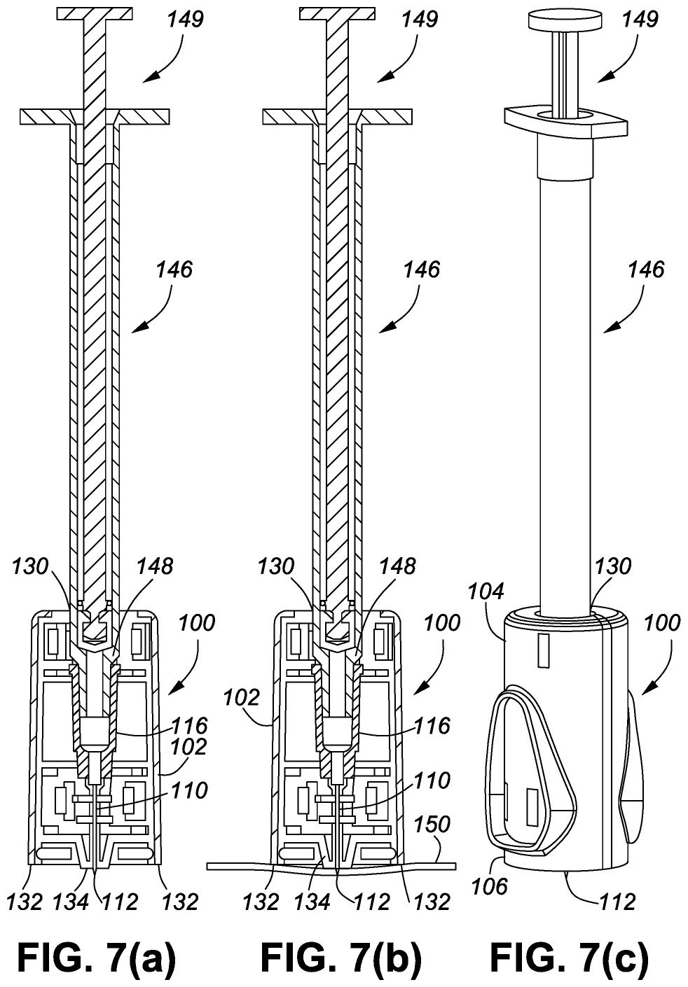

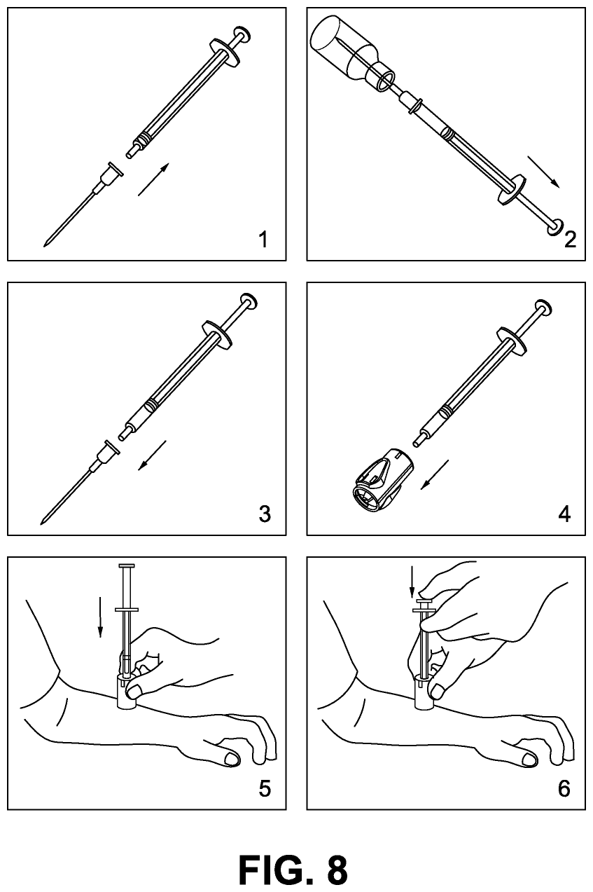

FIGS. 7(a) and (b) show a simplified cross-sectional view of an embodiment of the needle adaptor 100 engaged with a syringe 146, thus forming an injection device 149 for administering a fluid to a subject via injection. FIG. 7(c) illustrates a perspective view of the injection device 149 shown in FIGS. 7(a) and (b).

As shown in FIGS. 7(a) and (b), the tip 148 of the syringe (or another dosing device) can be inserted into the channel 130 disposed at the proximal end 104 of the housing 102 so as to engage the tip 148 with the needle hub 116. As shown in FIG. 7(b), the first contact surface 132 is engaged with the skin 150 of the subject. Pushing the housing 102 against the skin 150 allows the first end 112 of the needle shaft 110 (which extends from the second contact surface 134) to penetrate the skin 150. The fluid can then be expelled from the syringe 146 (or the other dosing device) through the needle shaft 110 into the subject.

FIG. 8 illustrates a series of steps that can be used in administering a fluid to a subject via injection using the needle adaptor 100. Following step 6, the syringe, used removable needle unit, and needle adaptor can be disposed of in an appropriate sharps container.

The above-described needle adaptor 100 is particularly suited for delivering multiple injections of a fluid to a subject. Multiple injections of a fluid may be desirable in certain applications, such as for stem cell transplants. The predefined distance d1 (penetration depth of the needle shaft 110) for such applications could be, for example, around 1.5 mm.

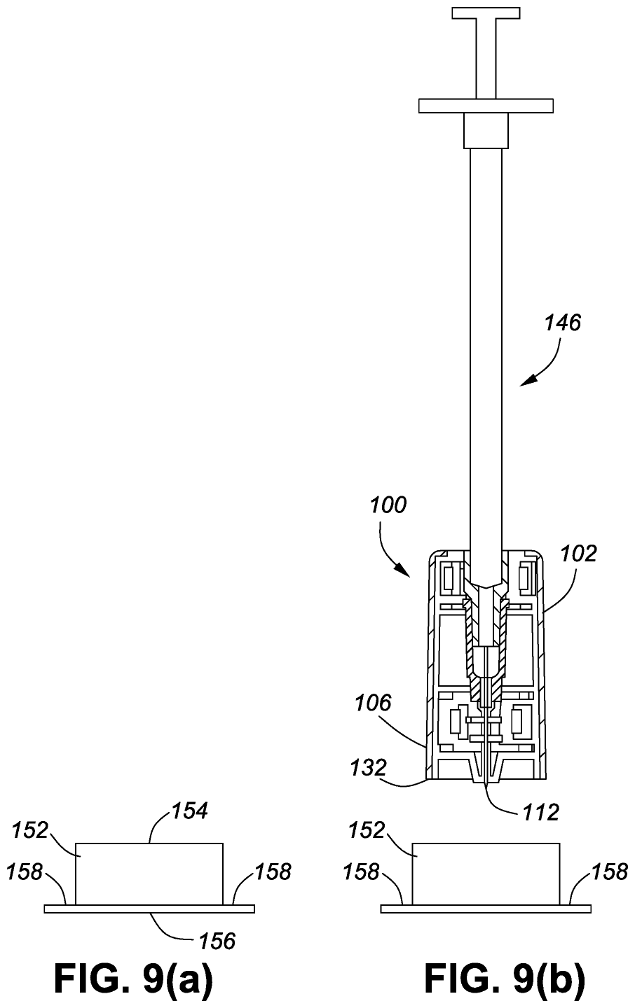

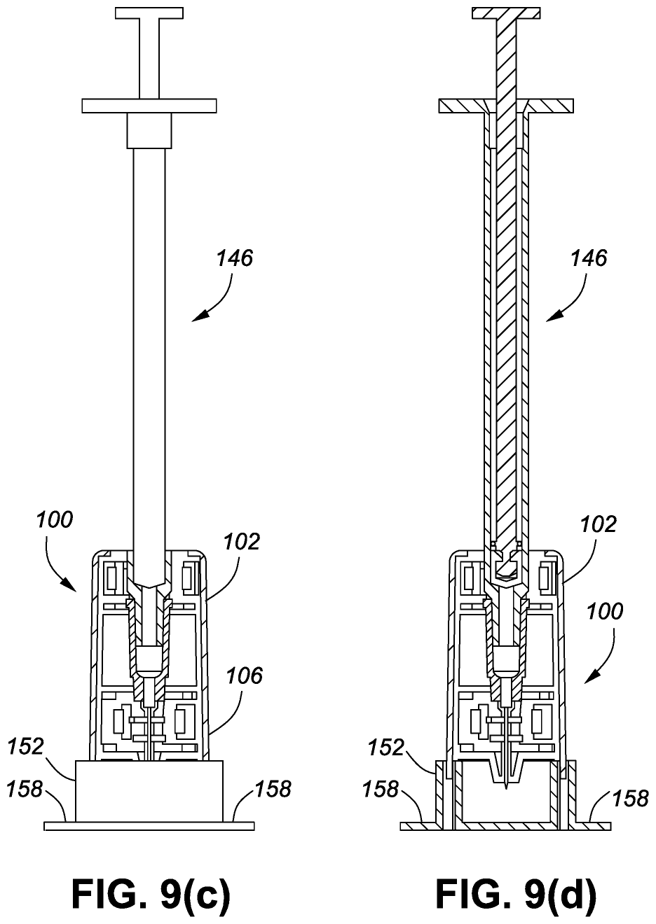

To increase safety, the needle adaptor housing 102 with the protruding first end 112 of the needle shaft 110 could be held in a safety holder 152 to prevent needle stick injuries when the device is not in use (e.g. before or after injection). FIGS. 9(a)-(d) illustrate a safety holder 152, wherein the safety holder 152 has an open end 154 for receiving at least the distal end 106 of the needle adaptor housing 102 and a closed end 156, the closed end 156 comprising opposed wings 158 for stabilizing the safety holder 152 on a horizontal surface. FIG. 9(a) illustrates the safety holder 152. FIG. 9(b) illustrates a simplified cross-sectional view of an embodiment of the needle adaptor 100 engaged with a syringe 146, and how the the distal end 106 of the needle adaptor housing 102 can be received in the open end 154 of the safety holder. FIG. 9(c) illustrates the needle adaptor housing 102 engaged with the safety holder 152, with the needle adaptor 100 in a simplified cross-sectional view. FIG. 9(d) illustrates the needle adaptor housing 102 engaged with the safety holder 152, with the needle adaptor 100 in a simplified cross-sectional view and with the safety holder 152 and syringe also in cross-sectional view. In the embodiment shown, the safety holder 152 includes an indentation around the perimeter of the open end 154 that is configured to engage with the first contact surface 132 of the needle adaptor housing 102.

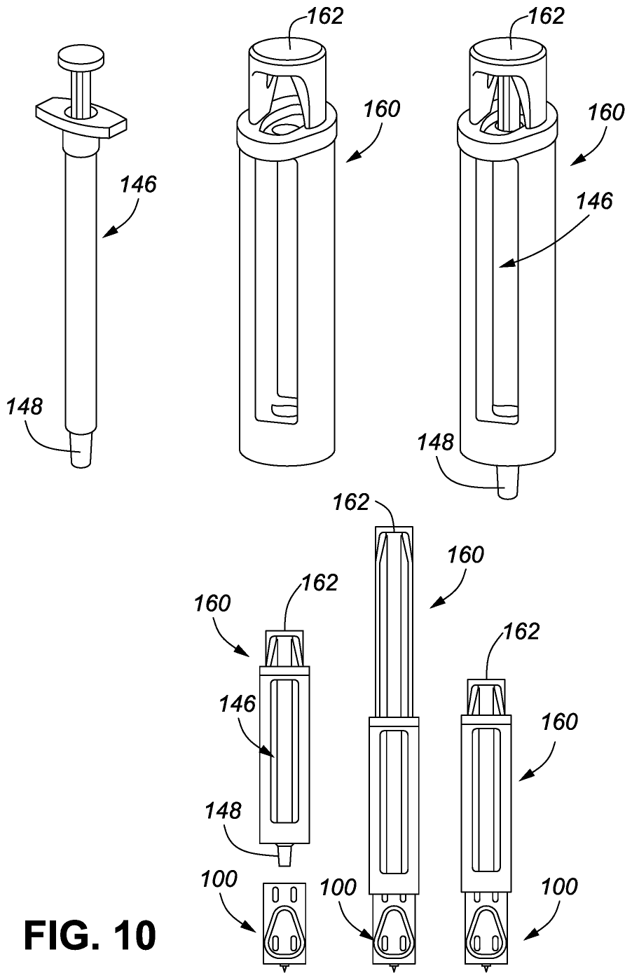

As noted above, the needle adaptor 100 can be coupled to a dose-metering device 160 that is compatible with a syringe 146, wherein the tip of the syringe 148 enters the channel 130 (not shown) of the needle adaptor 100 and the dose-metering device 160 has a plunger 162 that controls the amount of fluid being delivered in a single dose. This can allow for multiple dosed injections, such as between e.g. 0.01 or 0.2 mL, e.g. 0.05 mL. FIG. 10 illustrates the engagement of a syringe 146 with a dose-metering device 160, and the engagement of same with the needle adaptor 100. Fasteners such as push fittings or snaps could be used to effect stable engagement of the dose-metering device 160 and the needle adaptor 100. Alternatively or additionally, as noted above, other dosing devices besides a syringe can be engaged with the needle adaptor 100, wherein the other dosing devices comprise a dispensing tip that is similar in size and shape to a syringe tip. Such other dosing devices could include multi-chamber pre-filled containers (e.g. dual chambers for lyophilized substances and diluents), with a means for mixing the components of the chambers and means for expelling same from the dosing device (e.g. by way of a plunger, etc.).

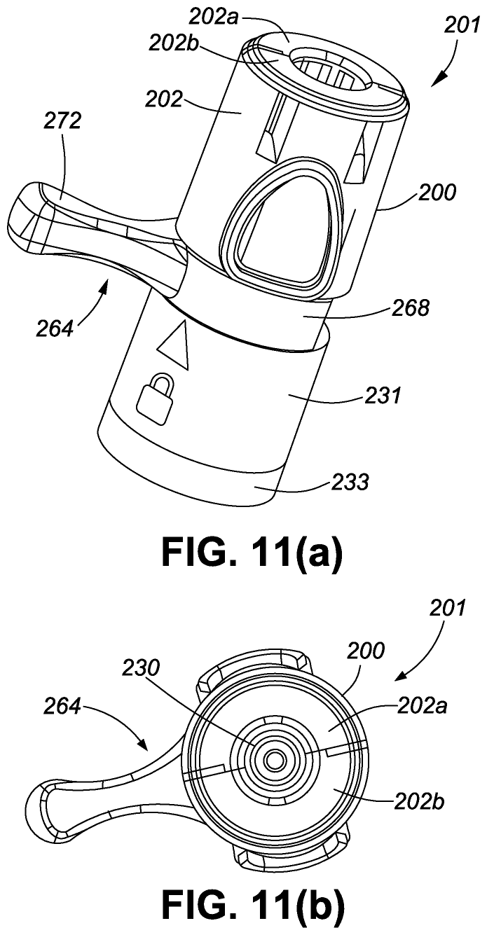

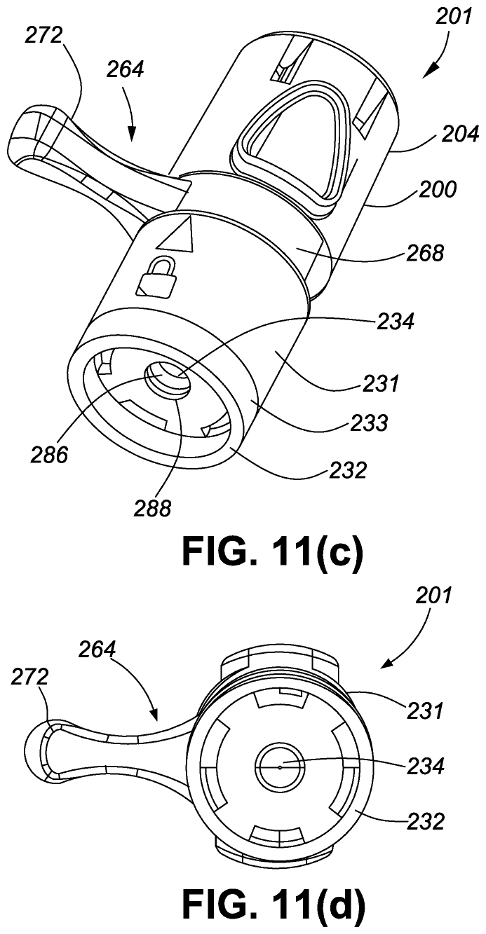

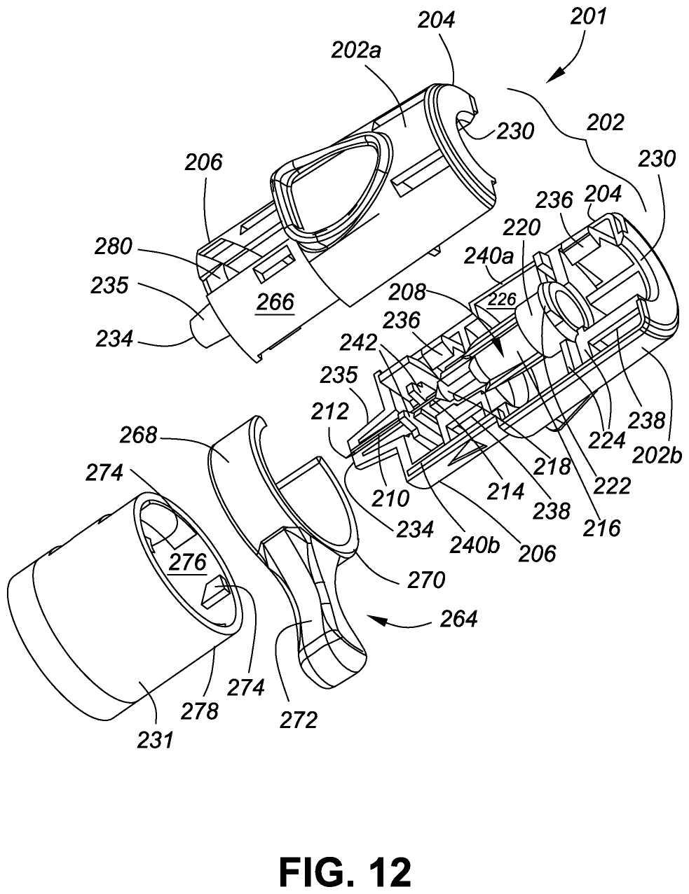

FIGS. 11(a) and (c) illustrate an assembly 201 for forming an injection device for administering a fluid to a subject in two slightly different perspective views. FIG. 11(b) illustrates a top view of the assembly shown in FIGS. 11(a) and (c), and FIG. 11(d) illustrates a bottom view of the assembly shown in FIGS. 11(a) and (c). FIG. 12 illustrates an exploded view of the assembly shown in FIGS. 11(a) and (c).

As can be seen from FIGS. 11(a)-(d) and FIG. 12, the assembly 201 comprises a foot 231 comprising a first contact surface 232 adapted to be placed on a skin of the subject, the foot 231 having a tubular shape for receiving a needle adaptor body 200.

As shown in FIG. 12, the needle adaptor body 200 comprises: a housing 202 formed from a first housing portion 202a and a second housing portion 202b, the housing 202 having a proximal end 204 and a distal end 206; and a needle unit 208 fixedly mounted within the housing 202. As with the above-described needle adaptor 100, in the present embodiment, the first housing portion 202a and the second housing portion 202b are of identical construction and can be formed using injection moulding of Medical Grade plastic (e.g. COC), which results in a very economical production. In the embodiment shown, the housing 202 is generally cylindrical in shape, and each of the first housing portion 202a and the second housing portion 202b is generally semi-cylindrical in shape.

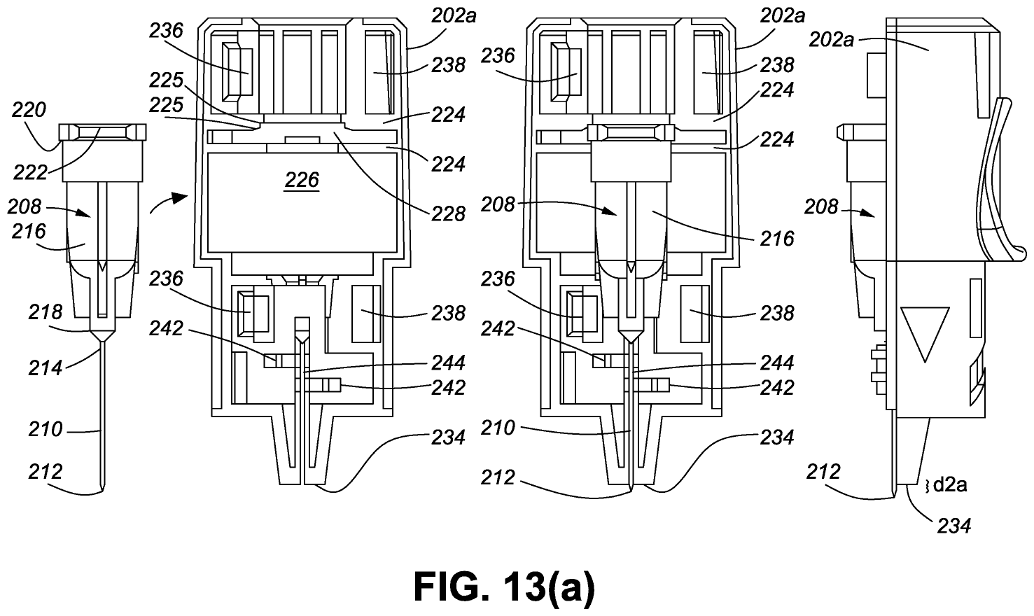

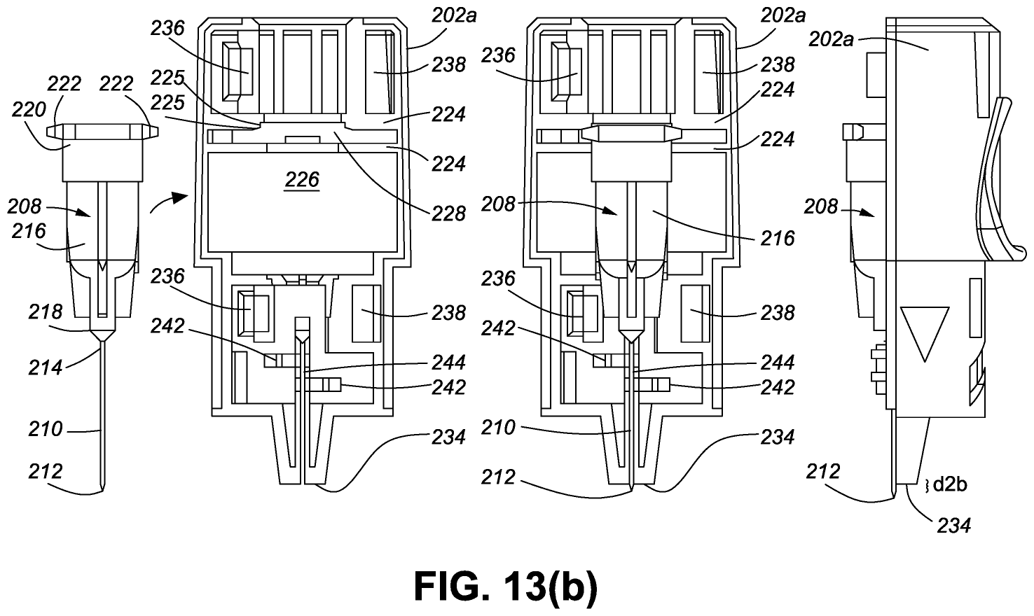

FIGS. 13(a) and 13(b) illustrate the first housing portion 202a and the manner in which the needle unit 208 can be engaged therewith, showing the first housing portion 202a in front and side view with the needle unit 208 engaged therewith. With reference to FIG. 12 and FIGS. 13(a) and (b), the needle unit 208 comprises: a needle shaft 210 comprising a first end 212 for penetrating the subject's skin and a second end 214 connected to a needle hub 216. The needle hub 216 comprises a distal end 218 connected to the second end 214 of the needle shaft 210 and a proximal end 220 comprising a pair of radially extending diametrically opposing flanges 222 (only one of which is visible in FIGS. 12 and 13(a)). In the embodiment shown, the needle hub 216 has typical needle hub tabs that would be found on commercially available needle units comprising a needle shaft and hub having a standard female Luer-Lok fitting.

Each of the first housing portion and the second housing portion comprises at least two consecutive transverse walls or projections 224 extending from an inner surface 226 thereof, wherein the at least two consecutive transverse walls or projections 224 form a gap 228 therebetween for receiving at least a portion of one or both of the pair of radially extending diametrically opposing flanges 222 of the needle unit 208 to fixedly mount the needle unit 208 within the housing 202. The proximal end 204 of the housing 202 together with the at least two consecutive transverse walls or projections 224 of each of the first housing portion 202a and the second housing portion 202b define a channel 230 for receiving a syringe tip for engagement with the needle hub 216. The distal end 206 of the housing 202 comprises a second contact surface 234, wherein the first end 212 of the needle shaft 210 extends out of the second contact surface 234 by a predefined distance d2 (e.g. d2a or d2b) for limiting a penetration depth of the needle shaft. In the embodiment shown, the first contact surface 232 is disposed along the perimeter of a distal end 233 of the foot 231, and the second contact surface 234 is substantially centrally disposed at the distal end 206 of the housing 202. Specifically, the second contact surface 234 is disposed at an end of a needle-stabilizing protrusion 235 which extends substantially centrally from the distal end 206 of the housing 202.

As will be described in further detail below, the needle adaptor body 200 is movably mounted to the foot 231 for allowing movement of the needle adaptor body 200 from a first position to a second position, wherein: when the needle adaptor body 200 is in the first position, the needle shaft 210 is in a retracted position such that the first end 212 of the needle shaft 210 does not extend beyond the first contact surface 232, and when the needle adaptor body 200 is in the second position, the first end 212 of the needle shaft 210 extends beyond the first contact surface 232 and out of the second contact surface 234 by the predefined distance d2 for limiting the penetration depth of the needle shaft. The assembly further comprising a friction means for inhibiting movement of the needle adaptor body 200 relative to the foot 231 when the needle adaptor body 200 is in the first position, until a predefined static friction force is overcome, and for causing or allowing a sudden acceleration of the needle adaptor body 200 towards the foot 231 for increasing a speed of the needle shaft 210 for increasing chance of penetration of the skin.

As noted above, FIGS. 13(a) and 13(b) illustrate the first housing portion 202a and the manner in which the needle unit 208 can be engaged therewith, namely in a first orientation as shown in FIG. 13(a), and a second orientation as shown in FIG. 13(b). In a similar manner as described above in respect of needle adaptor 100, varying the placement of the needle unit 208 within the first housing portion 202a impacts the predefined distance d2 that the first end of the needle shaft 212 extends out of the second contact surface 234. As noted above, as a result of current state-of-the-art manufacturing processes, it is known that final needle lengths will be subject to production tolerances of e.g. 0.05 mm, or e.g. 2 mm as defined in specific ISO standards. The needle adaptor body which forms part of the assembly described herein allows for control over penetration depth regardless of intended needle length and tolerance deviations. The assembly 201 of the present application therefore allows for the use of needle units having longer needle shafts, such as (pre-glued) commercially available needle units comprising a needle shaft and hub having a standard female Luer-Lok fitting, e.g. 26-34 G and 12 mm length. Such needle units can have long needle shafts with broad tolerances, while the assembly 201 of the present application can accurately control penetration depth irrespective of same, thereby accounting for and offsetting manufacturer variability in needle shafts.

As best seen in FIGS. 13(a) and (b), the gap 228 formed by the at least two consecutive transverse walls or projections 224 of the first housing portion 102a is configured to receive the at least a portion of one or both of the pair of radially extending diametrically opposing flanges 222 of the needle unit 208 in one of a first orientation (FIG. 13(a)) and a second orientation (FIG. 13(b)) of mounting of the needle unit 208. As best seen in FIGS. 13(a) and 13(b), the first and second orientation of mounting of the needle unit can differ from one another by about a 90 degree rotation. Stepped stops or shoulders 225 are present in at least one of the at least two consecutive transverse walls or projections 224 of the first housing portion 202a, which engage with the distal end 218 of the needle hub 216 and facilitate fixedly mounting the needle unit 208 in the first orientation or the second orientation. The flanges 222 of the needle unit 208 have a frictional engagement with opposing surfaces of the at least two consecutive transverse walls or projections 224 of each of the first housing portion 202a and the second housing portion 202b when received in the gap 228 therebetween, thus avoiding the need to use less desirable means for fixedly engaging the needle unit within the housing, such as gluing, overmoulding, etc. as discussed above.

With continued reference to FIGS. 13(a) and (b), the predefined distance d2 by which the first end 212 of the needle shaft 210 extends out of the second contact surface 234 is a first predefined distance d2a when the needle unit 208 is mounted in the first orientation (i.e. when the distal end 218 of the needle hub 216 is oriented with the pair of radially extending diametrically opposing flanges 222 extending directly into, and out of, the plane of the paper in FIG. 13(a)—this is also referred to herein as the flanges being disposed in a “vertical” position, or Position V). As best shown in FIG. 13(b), the predefined distance d2 by which the first end 212 of the needle shaft 210 extends out of the second contact surface 234 is a second predefined distance d2b when the needle unit 208 is mounted in the second orientation (i.e. when the distal end 218 of the needle hub 216 is oriented with the pair of radially extending diametrically opposing flanges 222 extending from side-to-side as shown in FIG. 13(b)—this is also referred to herein as the flanges being disposed in a “horizontal” position, or Position H). As illustrated in FIGS. 13(a) and 13(b), the first predefined distance d2a is different from the second predefined distance d2b. Again, while the above-noted description is with reference to the first housing portion 202a, it will be understood that the first housing portion 202a is interchangeable with the second housing portion 202b, given they are of identical construction.

As noted above, FIG. 5 represents a simplified first housing position 202a of the needle adaptor body 200 of the assembly 201 discussed in further detail above. In the labels for FIG. 5 and in the above description of same, references to components/elements of the needle adaptor body 200 of the assembly 201 are provided in brackets. This disclosure will not be repeated here, for conciseness. However, referring to FIG. 5, and the accompanying description of same as well as Table 1, it will be clear to the skilled worker that the assembly of the present application has a very significant benefit in that it can account for and offset manufacturer variability in needle shafts.

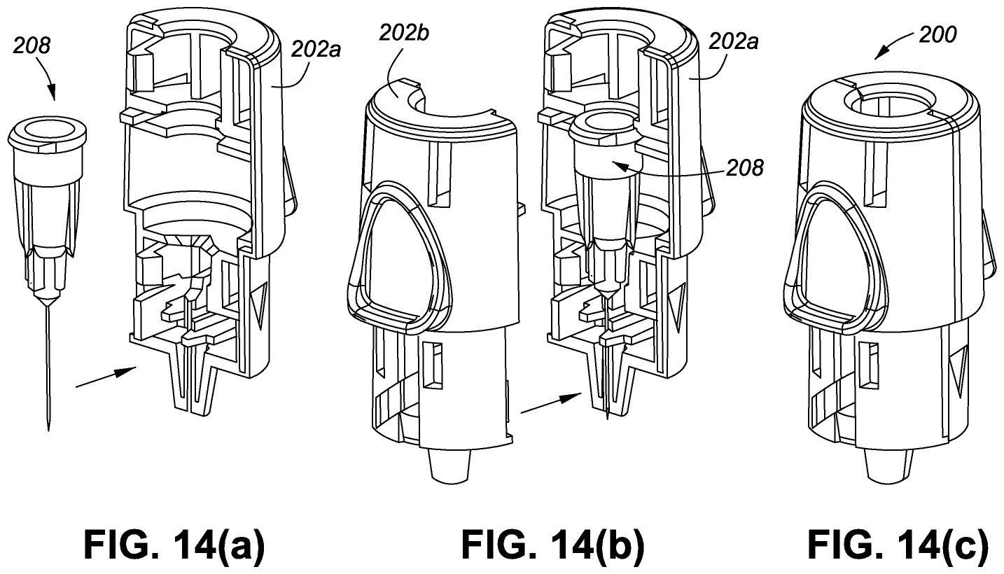

As shown in FIGS. 14(a)-(c), following engagement of the needle unit 208 with the first housing portion 202a (FIG. 14(a)), the second housing portion 202b is then engaged with the first housing portion 202a (FIG. 14(b)) to form the needle adaptor body 200 (FIG. 14(c)). As the skilled worker will appreciate, it is equally possible for the needle unit to engage with the second housing portion 202b in the same manner as shown for the first housing portion 202a in FIGS. 14(a)-(c), given the first housing portion 202a and the second housing portion 202b are of identical construction.

As can be seen from FIGS. 12-14, the first housing portion 202a and the second housing portion 202b are configured for snap-fit engagement with one another to form the housing 202. This is accomplished by way of fasteners (i.e. snaps) 236 which project from the inner surface of each of the first housing portion 202a and the second housing portion 202b, which are configured to engage with complementary slots 238 formed within an inner portion of the housing of each of the first housing portion 202a and the second housing portion 202b in a snap-fit engagement. Each of the first housing portion 202a and the second housing portion 202b also has complementary ribs 240a and 240b which further assist engagement of the first housing portion 202a and the second housing portion 202b to form the housing 202. As such, the snap-fit engagement of the first housing portion 202a and the second housing portion 202b to form the housing 202 is a simple and straightforward means for fixedly joining these two components of the housing 202 together, which does not require the use of glue or other means for joining these components.

Referring to FIGS. 12-14, each of the first housing portion 202a and the second housing portion 202b further comprises a plurality of projections 242 extending from the inner surface 226 of a distal end thereof to form a needle guide 244 configured to hold the needle shaft 210 in place. In the embodiment shown, the plurality of projections comprises at least two needle-stabilizing projections 242 disposed on either side of the needle shaft 210 and offset from one another along a longitudinal axis of the needle shaft 210, each of the at least two needle-stabilizing projections 242 having a sloped surface abutting the needle shaft. These features of the needle adaptor stabilize the needle shaft and hold it in a fixed position upon assembly of the first housing portion 202a and the second housing portion 202b to form the housing 202.

As with the above-described needle adaptor 100, the angle between a longitudinal axis of the needle shaft 210 and a tangential plane defined by the first contact surface 232 is about 90°. It will be understood by the skilled worker that this angle can be varied (e.g. to be in the range of, for example, from about 5° to about 175°, from about 10° to about 170°, from about 60° to about 120°, for example from about 80° to about 100°) by adjusting the angle by which the at least two consecutive transverse walls or projections 224 extend from the inner surface 226 of the first/second housing portions (202a/202b) along with the positioning of other supporting features (e.g. needle guide 244) and of the foot 231, etc

As noted above, the needle adaptor body 200 is movably mounted to the foot 231 for allowing movement of the needle adaptor body 200 from a first position to a second position. In the embodiment shown in FIGS. 11(a)-(d) and FIG. 12, a locking mechanism is present for providing a locked mode and an unlocked mode of the device, the locked mode being a mode of the assembly, wherein the needle adaptor body 200 is prevented from moving axially towards the foot 231, even when an axial force larger than the predefined static friction is exerted on the needle adaptor body 200 relative to the foot 231; the unlocked mode being a mode of the assembly wherein the needle adaptor body 200 is allowed to move towards the foot 231, when an axial force larger than the predefined static friction is applied to the needle adaptor body 200 relative to the foot 231. The locking mechanism shown in FIGS. 11(a)-(d) and FIG. 12 comprises a removable safety clip 264 configured to engage with a portion of the outer surface 266 of the housing 202 to maintain the foot 231 and needle adaptor body 200 spaced apart from one another to prevent the needle adaptor body 200 from moving axially towards the foot 231. The removable safety clip 264 is formed from a resilient material (e.g. Medical Grade plastic) and has a first leg 268 and a second leg 270 extending from a handle or grip 272, wherein the first leg 268 and the second leg 270 define a general C-shape for engaging with the outer surface 266 of the housing 202. Other, alternative locking mechanisms are known in the art, such as those disclosed in WO2017168015(A1)—e.g. the assembly could be unlocked when the foot is rotated relative to the needle adaptor body around the longitudinal axis, resulting in an assembly in the “unlocked state”, thus permitting the needle adaptor body to move toward the foot.

As also noted above, the assembly comprises a friction means for inhibiting movement of the needle adaptor body 200 relative to the foot 231 when the needle adaptor body 200 is in the first position, until a predefined static friction force is overcome, and for causing or allowing a sudden acceleration of the needle adaptor body 200 towards the foot 231 for increasing a speed of the needle shaft 210 for increasing chance of penetration of the skin. The predefined static friction force can be a value in the range from about 1.0 to about 20.0 Newton, or from about 1.5 to about 15 Newton, or from about 2.0 to 1 about 0 Newton, or from about 5.0 to about 7.5 Newton; preferably the static friction force is at least about 2.0 Newton.

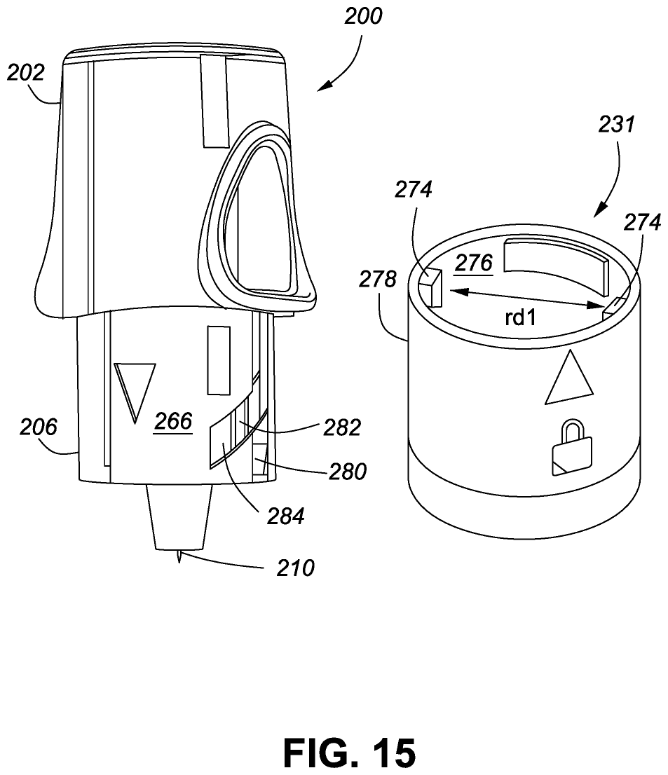

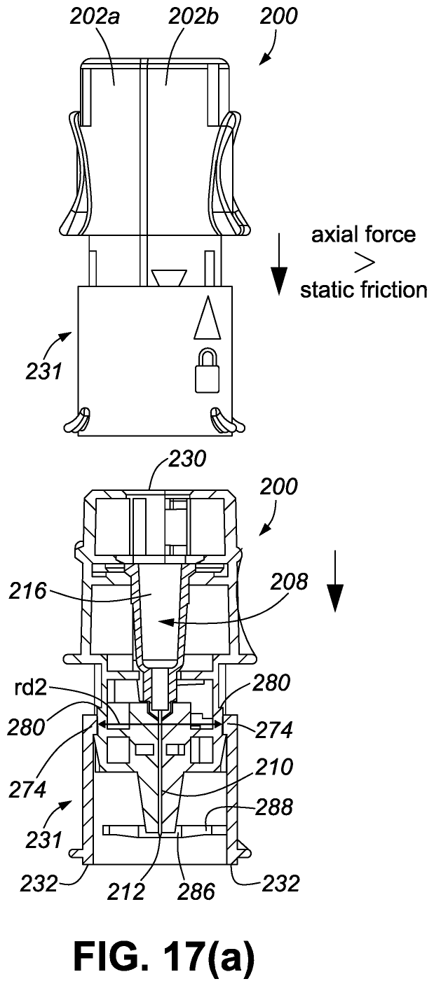

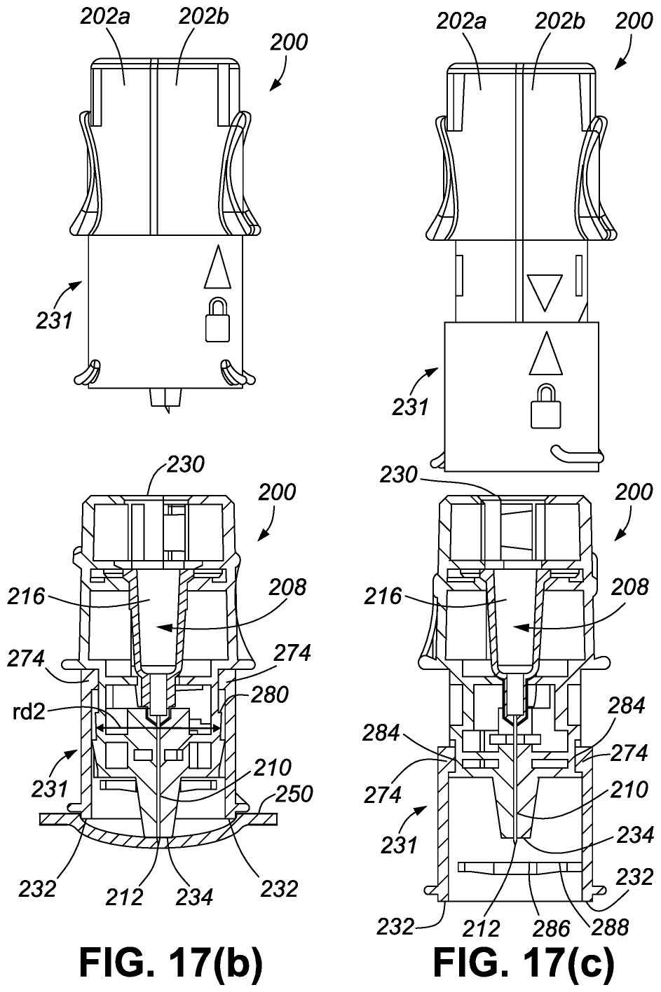

In the embodiment shown in FIG. 12, the friction means comprises at least two protrusions 274 extending from an inner surface 276 of a proximal end 278 of the foot 231 being in contact with at least two corresponding grooves 280 located on an outer surface 266 of the distal end 206 of the housing 202 of the needle adaptor body 200, wherein a radial dimension rd1 defined by the at least two protrusions 274 before assembly of the needle adaptor body 200 and the foot 231 (see FIG. 15), is smaller than a radial dimension rd2 defined by the at least two corresponding grooves 280 (see FIG. 17(a)-(b)), the static friction being provided by radial clamping. This is described in further detail below. The at least two corresponding grooves 280 are oriented generally parallel to a longitudinal axis of the housing 202.

It will be understood that the first friction means could equivalently comprise at least two protrusions extending from an outer surface of the body being in contact with at least two corresponding grooves located on an inner surface of the foot, wherein a radial dimension defined by the at least two protrusions before assembly of the body and the foot, is larger than a radial dimension defined by the grooves, the static friction being provided by radial clamping.

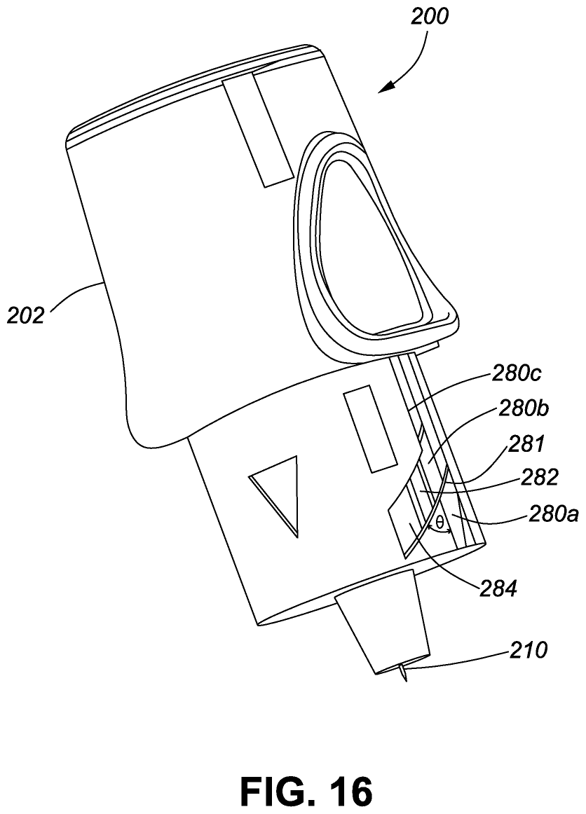

FIG. 15 illustrates a perspective view of the needle adaptor body 200 and foot 231, where the at least two protrusions 274 extending from an inner surface 276 of a proximal end 278 of the foot 231 and one of the at least two corresponding grooves 280 located on an outer surface 266 of the distal end 206 of the housing 202 of the needle adaptor body 200 can be clearly seen. FIG. 16 provides an enlarged perspective view of the needle adaptor body 200 to better illustrate the contours of groove 280.

As can be best seen in FIG. 16, the at least two corresponding grooves 280 (one shown) are configured to prevent disengagement of the foot 231 from the needle adaptor body 200 by limiting movement of the foot 231 away from the needle adaptor body 200 following engagement of the at least two protrusions 274 extending from the inner surface 276 of the proximal end 278 of the foot 231 with the at least two corresponding grooves 280. Groove portion 280a of each of the at least two corresponding grooves 280 is angled slightly towards the center of needle adaptor body 200 to facilitate initial engagement of the at least two protrusions 274 with the at least two corresponding grooves 280. However, once the at least two protrusions 274 engage with the at least two corresponding grooves 280 and move towards the distal end 206 of housing 202 to reach groove portion 280b of each of the at least two corresponding grooves 280, it can be seen that movement of the foot 231 away from the needle adaptor body 200 is limited by a ridge 281 formed between groove portion 280b and groove portion 280a (which projects upward from groove portion 280b at an angle of about 90 degrees). At this point, the static friction is provided by radial clamping as noted above. When the locking mechanism is disengaged and when predefined static friction force is overcome—i.e. when an axial force larger than the predefined static friction is exerted on the needle adaptor body relative to the foot—the at least two protrusions 274 move towards groove portion 280c of each of the at least two corresponding grooves 280, where the friction between the surfaces undergoes a sudden decrease and/or drops to zero. This causes or allows a sudden acceleration of the needle adaptor body 200 towards the foot 231 for increasing a speed of the needle shaft 210 for increasing chance of penetration of the skin.

As shown in FIGS. 15 and 16, the assembly further comprises at least two deactivation grooves 282 located on the outer surface 266 of the distal end 206 of the housing 202 of the needle adaptor body 100, wherein each of the at least two deactivation grooves 282 intersects one of the at least two corresponding grooves 280 at an angle theta (θ) (about 25° to about 65°, e.g. about 45°) relative to the longitudinal axis of the housing 202, such that axial movement of the foot 231 away from the needle adaptor body 200 and rotation of the foot 231 relative to the needle adaptor body 200 engages the at least two protrusions 274 with the at least two deactivation grooves 282 (one shown), wherein each of the at least two deactivation grooves 282 comprises an indentation 284 complementary to a shape of each of the at least two protrusions 274 to fixedly engage each of the at least two protrusions 274, such that the needle adaptor body 200 is held in a fixed, deactivated position relative to the foot 231, wherein the first end 212 of the needle shaft 210 does not extend beyond the first contact surface 232 when the needle adaptor body 200 is in the fixed, deactivated position relative to the foot 231.

FIG. 17(a) illustrates an embodiment of an assembly 201 in perspective (upper) and cross-sectional view (lower) wherein the safety clip 264 has been removed and the needle adaptor body 200 is in the first position (i.e. ready for injection). FIG. 17(b) illustrates assembly 201 in perspective (upper) and cross-sectional view (lower; showing the skin 250 of the subject) wherein the safety clip 264 has been removed and the needle adaptor body 200 is in the second position (i.e. the needle penetrates the skin). FIG. 17(c) illustrates assembly 201 in perspective (upper) and cross-sectional view (lower) wherein the needle adaptor body 200 is held in a fixed, deactivated position relative to the foot 231 (from the second position, involves axial movement of foot 231 away from needle adaptor body 200 and rotation of foot 231 relative to needle adaptor body 200).

As shown in FIGS. 17(a)-(c), movement of the device from the first position to the second position causes or allows a sudden acceleration of the needle adaptor body 200 towards the foot 231, and the needle-stabilizing protrusion 235 passes through an aperture 286 formed by an interior surface 288 of the foot 231 as it accelerates towards the skin 250.

As noted above, the features of the needle adaptor body 200 of the assembly 201 can account for and offset manufacturer variability in needle shafts by having the ability to mount the needle unit 208 in two different orientations within the housing 202. If it is necessary to further adjust the predefined distance d2 that the first end 212 of the needle shaft 210 extends from the second contact surface 234, this can be done during assembly of the device. A method for assembling the assembly can therefore comprise: obtaining the foot 231; obtaining the first housing portion 202a and the second housing portion 202b forming the housing 202 of the needle adaptor body 200; obtaining the needle unit 208; obtaining the removable safety clip 264; measuring a length of the needle shaft 210; determining whether the needle unit 208 is to be mounted in the first orientation or the second orientation based on the length of the needle shaft 210; optionally, removing a preselected portion p2 of a distal end of each of the first housing portion 202a and the second housing portion 202b based on the length of the needle shaft 210 and based on whether the needle unit 208 is to be mounted in the first orientation or the second orientation; mounting the needle unit 208 in one of the first housing portion 202a and the second housing portion 202b in the first orientation or the second orientation by inserting the at least a portion of one or both of the pair of radially extending diametrically opposing flanges 222 of the needle unit 208 into the gap 228 formed between the at least two consecutive transverse walls or projections 224; and engaging the first housing portion 202a and the second housing portion 202b with one another to form the housing 202 of the needle adaptor body 200; engaging the removable safety clip 264 with the portion of the outer surface 266 of the housing 202; and engaging the foot 231 and the needle adaptor body 200. Engaging the foot 231 and the needle adaptor body 200 comprises engaging the at least two protrusions 274 extending from the inner surface 276 of the proximal end 278 of the foot 231 with the at least two corresponding grooves 280 located on the outer surface 266 of the distal end 206 of the housing 202 of the needle adaptor body 200. The preselected portion p2 of a distal end of each of the first housing portion 202a and the second housing portion 202b can be removed from the needle-stabilizing protrusion 235, in a similar manner as shown in FIG. 6 in respect of the needle adaptor 100 described above. Removing the preselected portion p2 of the distal end of each of the first housing portion 202a and the second housing portion 202b can comprise cutting the preselected portion p2 of the distal end of each of the first housing portion 202a and the second housing portion 202b, such as by laser cutting.

The assembly process can further be automated such that a vision/imaging system (“Machine Vision”) on an automated assembly line (e.g. based on CCD cameras) incorporating “pick and place” robotics technology can be used to assemble the device. The general assembly process can proceed as follows:

1. Pre-manufactured components: Housing shell—i.e. first and second housing portions 202a and 202b (2x, injection moulded); Safety clip 264 (injection moulded); Foot 231 (injection moulded); Needle unit 208 (preferably obtained from a Food and Drug Administration (FDA)-approved source)

2. Feeding components in the system (manually or (semi-)automatically): In feeders (e.g. for the injection moulded components); In trays or racks (e.g. for the needle units)

The following Step 3 or 4 can run in parallel or in random orders with respect to each other.

3. An imaging system measures the exact length of the needle shaft 112 (e.g. with 0.005 mm accuracy)

4. 2 housing shells (202a and 202b) are prepared/fed into the automated system.

Step 5 is optional

5. The needle-stabilizing protrusion 235 of both housing shells 202a and 202b can be lasered to improve the final needle shaft length for skin penetration)

6. The needle is placed into 1 housing shell (202a), e.g. wings horizontal, or e.g. wings vertical to compensate for length deviations from step 3.

7. The second (e.g. identical) housing shell (202b) is (e.g. automatically) mounted (e.g. snapped on).

Step 8 is optional/quality related

8. Perform (imaging) measurements of the residual (penetration) length of the needle shaft.

9. The safety clip 264 is installed

10. The foot 231 is installed

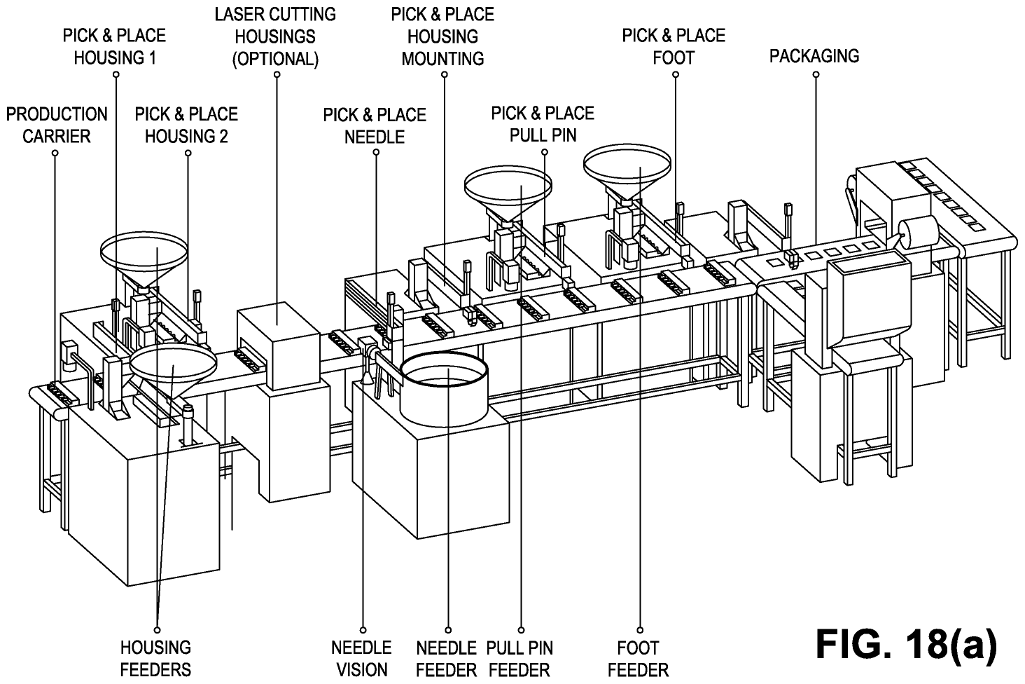

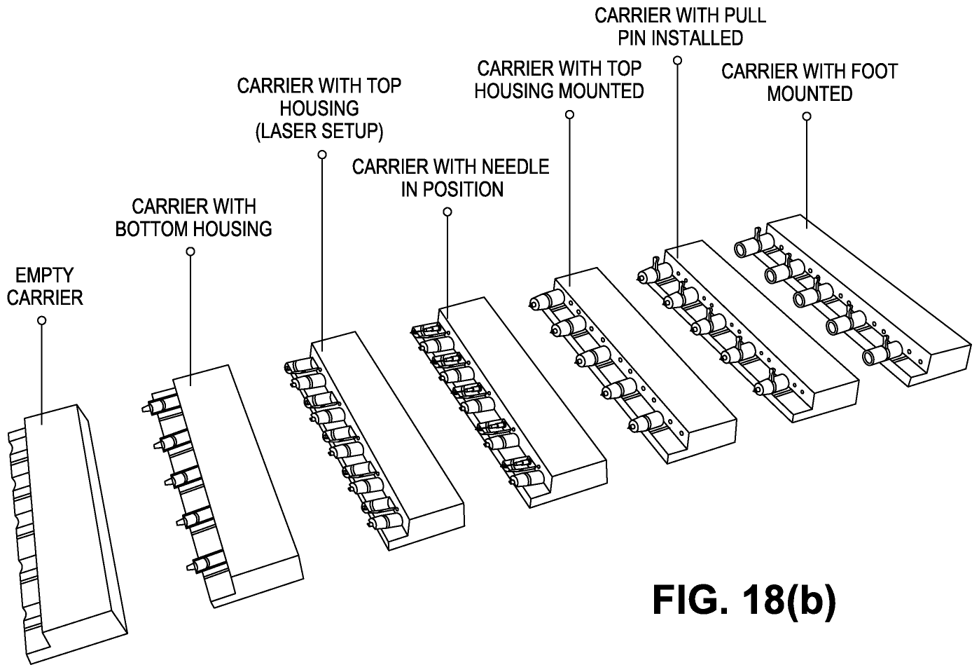

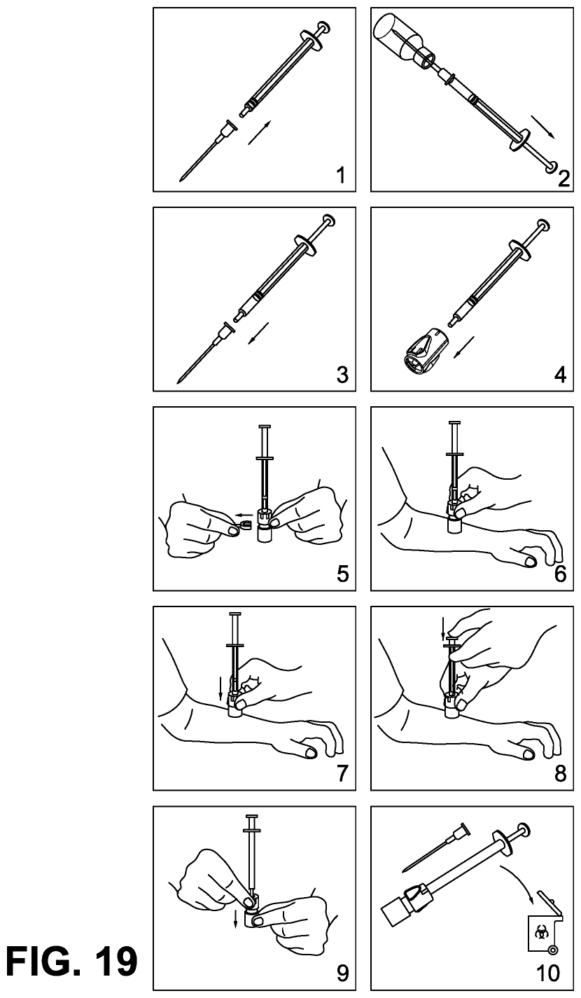

FIG. 18(a) illustrates a proposed automatic assembly line for preparing assembly 201 using Machine Vision and pick-and-place robotics technology. “Housing 1” and “Housing 2” refer to first and second housing portions 202a and 202b, “Needle” refers to needle unit 208, “Housing Mounting” refers to mounting needle unit 208 in one of the first and second housing portions 202a/202b, “Pull Pin” refers to the safety clip 264, and “Foot” refers to foot 231. The various elements are placed in the production carrier which moves along the assembly line via a conveyer belt. FIG. 18(b) illustrates the production carrier makeup at each stage of the assembly. Components can be held in the carrier via engagement of slots that can be built in during the injection moulding of same, and/or can be held in place via a light vacuum or other means known to those of skill in the art.

As the skilled worker will appreciate, it is highly convenient to be able to manufacture the assembly 201 from injection moulded, pre-manufactured components that can be assembled via snap-fit engagement (as opposed to the use of glue or other attachment methods). Furthermore, the ability to automate manufacture of the assembly 201 greatly reduces production costs. However, it is of course possible to manufacture the assembly 201 manually as well.

A method of administering a fluid to a subject via injection using the assembly 201 can comprise: (a) obtaining the assembly 201, wherein the needle adaptor body 200 is in the first position; (b) obtaining a syringe 246 or other dosing device, wherein the other dosing device comprises a dispensing tip that is similar in size and shape to a syringe tip, wherein the syringe 246 or the other dosing device is loaded with the fluid to be administered to the subject; (c) inserting the tip 248 of the syringe 246 or the other dosing device into the channel 230 disposed at the proximal end 204 of the needle adaptor housing 202 so as to engage the tip with the needle hub 216; (d) engaging the first contact surface 232 of the foot 231 with the skin of the subject; (e) pushing the housing 202 of the needle adaptor body 200 towards the foot 231 in an axial direction to move the needle adaptor body 200 from the first position to the second position, thus causing the first end 212 of the needle shaft 210 to penetrate the skin; (f) expelling the fluid from the syringe 246 or the other dosing device through the needle shaft 210 into the subject; and (g) pulling the housing 202 of the needle adaptor body 200 away from the foot 231 in an axial direction and rotating the foot 231 relative to the needle adaptor body 200 to engage the at least two protrusions 274 with the at least two deactivation grooves 284 and to fixedly engage each of the at least two protrusions 274 in the indentation 284 in each of the at least two deactivation grooves 282, such that the needle adaptor body 200 is held in the fixed, deactivated position relative to the foot; the method further comprising removing the safety clip 264 from the outer surface 266 of the housing 202 after step (c) and prior to step (d), or after step (d) and prior to step (e). As noted above, other dosing devices could include multi-chamber pre-filled containers (e.g. dual chambers for lyophilized substances and diluents), with a means for mixing the components of the chambers and means for expelling same from the dosing device (e.g. by way of a plunger, etc.).

FIG. 19 illustrates a series of steps that can be used in administering a fluid to a subject via injection using the assembly 201. As noted above, the assembly 201 described herein is particularly suitable for single use.

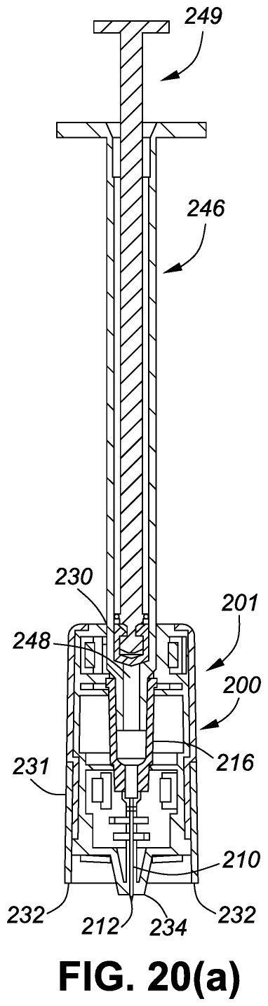

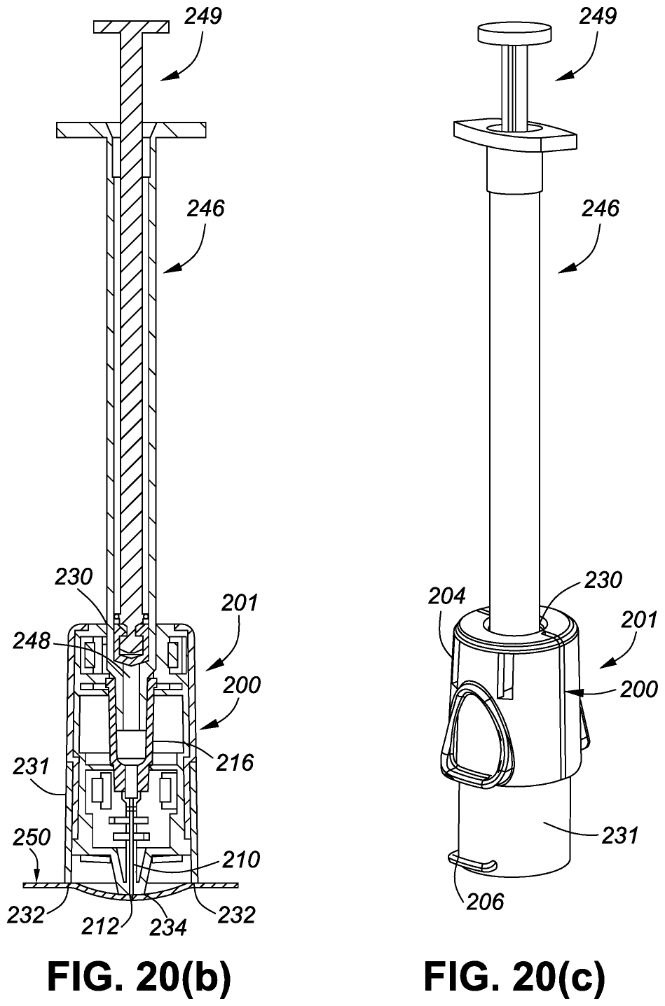

FIGS. 20(a) and (b) show a simplified cross-sectional view of an embodiment of the assembly 201 engaged with a syringe 246, thus forming an injection device 249 for administering a fluid to a subject via injection. As can be seen from FIGS. 20(a) and (b), the locking mechanism is absent and the needle adaptor body 200 is in the second position wherein the needle shaft 210 penetrates the skin 250 (shown in FIG. 20(b)). FIG. 20(c) illustrates a perspective view of the injection device 249 shown in FIGS. 20(a) and (b).

Although the present invention has been described with reference to the preferred embodiments, it is to be understood that modifications and variations may be resorted to without departing from the spirit and scope of the invention, as those skilled in the art readily understand. Such modifications and variations are considered to be within the purview and scope of the invention and the appended claims.

1. A needle adaptor for forming an injection device for administering a fluid to a subject comprising:
a housing formed from a first housing portion and a second housing portion, the housing having a proximal end and a distal end;
a needle unit fixedly mounted within the housing, wherein the needle unit comprises:
a needle shaft comprising a first end for penetrating the subject's skin and a second end connected to a needle hub,
the needle hub comprising a distal end connected to the second end of the needle shaft and a proximal end comprising a pair of radially extending diametrically opposing flanges;
wherein each of the first housing portion and the second housing portion comprises at least two consecutive transverse walls or projections extending from an inner surface thereof, wherein the at least two consecutive transverse walls or projections form a gap therebetween for receiving at least a portion of one or both of the pair of radially extending diametrically opposing flanges of the needle unit to fixedly mount the needle unit within the housing;
wherein the proximal end of the housing together with the at least two consecutive transverse walls or projections of each of the first housing portion and the second housing portion define a channel for receiving a syringe tip for engagement with the needle hub;
wherein the distal end of the housing comprises a first contact surface adapted to be placed on a skin of the subject and a second contact surface, wherein the first end of the needle shaft extends out of the second contact surface by a predefined distance for limiting a penetration depth of the needle shaft.

2. The needle adaptor of claim 1, wherein the gap formed by the at least two consecutive transverse walls or projections of each of the first housing portion and the second housing portion is configured to receive the at least a portion of one or both of the pair of radially extending diametrically opposing flanges of the needle unit in one of a first orientation and a second orientation of mounting of the needle unit,
wherein:
the predefined distance by which the first end of the needle shaft extends out of the second contact surface is a first predefined distance when the needle unit is mounted in the first orientation, and
the predefined distance by which the first end of the needle shaft extends out of the second contact surface is a second predefined distance when the needle unit is mounted in the second orientation,
wherein the first predefined distance is different from the second predefined distance.

3. The needle adaptor of claim 1 or 2, wherein the at least a portion of one or both of the pair of radially extending diametrically opposing flanges of the needle unit has a frictional engagement with opposing surfaces of the at least two consecutive transverse walls or projections of each of the first housing portion and the second housing portion when received in the gap therebetween.

4. The needle adaptor of any one of claims 1-3, wherein the first housing portion and the second housing portion are configured for snap-fit engagement with one another to form the housing.

5. The needle adaptor of any one of claims 1-4, wherein each of the first housing portion and the second housing portion further comprises a plurality of projections extending from the inner surface of a distal end thereof to form a needle guide configured to hold the needle shaft in place.

6. The needle adaptor of claim 5, wherein the plurality of projections comprises at least two needle-stabilizing projections disposed on either side of the needle shaft and offset from one another along a longitudinal axis of the needle shaft, each of the at least two needle-stabilizing projections having a sloped surface abutting the needle shaft.

7. The needle adaptor of any one of claims 1-6, wherein the first contact surface is disposed along the perimeter of the distal end of the housing, and the second contact surface is substantially centrally disposed at the distal end of the housing.

8. The needle adaptor of any one of claims 1-7, wherein the first housing portion and the second housing portion are of at least substantially similar or identical construction.

9. The needle adaptor of any one of claims 1-8, wherein the housing is generally cylindrical in shape.

10. The needle adaptor of any one of claims 1-9, wherein each of the first housing portion and the second housing portion is generally semi-cylindrical in shape.

11. An assembly for forming an injection device for administering a fluid to a subject, the assembly comprising:
a foot comprising a first contact surface adapted to be placed on a skin of the subject, the foot having a tubular shape for receiving a needle adaptor body;
a needle adaptor body comprising:
a housing formed from a first housing portion and a second housing portion, the housing having a proximal end and a distal end;
a needle unit fixedly mounted within the housing, wherein the needle unit comprises:
a needle shaft comprising a first end for penetrating the subject's skin and a second end connected to a needle hub,
the needle hub comprising a distal end connected to the second end of the needle shaft and a proximal end comprising a pair of radially extending diametrically opposing flanges;
wherein each of the first housing portion and the second housing portion comprises at least two consecutive transverse walls or projections extending from an inner surface thereof, wherein the at least two consecutive transverse walls or projections form a gap therebetween for receiving at least a portion of one or both of the pair of radially extending diametrically opposing flanges of the needle unit to fixedly mount the needle unit within the housing;
wherein the proximal end of the housing together with the at least two consecutive transverse walls or projections of each of the first housing portion and the second housing portion define a channel for receiving a syringe tip for engagement with the needle hub; and
wherein the distal end of the housing comprises a second contact surface, wherein the first end of the needle shaft extends out of the second contact surface by a predefined distance for limiting a penetration depth of the needle shaft;
the needle adaptor body being movably mounted to the foot for allowing movement of the needle adaptor body from a first position to a second position, wherein:
when the needle adaptor body is in the first position, the needle shaft is in a retracted position such that the first end of the needle shaft does not extend beyond the first contact surface, and
when the needle adaptor body is in the second position, the first end of the needle shaft extends beyond the first contact surface and out of the second contact surface by the predefined distance for limiting the penetration depth of the needle shaft;
the assembly further comprising a friction means for inhibiting movement of the needle adaptor body relative to the foot when the needle adaptor body is in the first position, until a predefined static friction force is overcome, and for causing or allowing a sudden acceleration of the needle adaptor body towards the foot for increasing a speed of the needle shaft for increasing chance of penetration of the skin.

12. The assembly of claim 11, wherein the gap formed by the at least two consecutive transverse walls or projections of each of the first housing portion and the second housing portion is configured to receive the at least a portion of one or both of the pair of radially extending diametrically opposing flanges of the needle unit in one of a first orientation and a second orientation of mounting of the needle unit,
wherein:
the predefined distance by which the first end of the needle shaft extends out of the second contact surface is a first predefined distance when the needle unit is mounted in the first orientation, and
the predefined distance by which the first end of the needle shaft extends out of the second contact surface is a second predefined distance when the needle unit is mounted in the second orientation,
wherein the first predefined distance is different from the second predefined distance.

13. The assembly of claim 11 or 12, wherein the at least a portion of one or both of the pair of radially extending diametrically opposing flanges of the needle unit has a frictional engagement with opposing surfaces of the at least two consecutive transverse walls or projections of each of the first housing portion and the second housing portion when received in the gap therebetween.

14. The assembly of any one of claims 11-13, wherein the first housing portion and the second housing portion are configured for snap-fit engagement with one another to form the housing.

15. The assembly of any one of claims 11-14, wherein each of the first housing portion and the second housing portion further comprises a plurality of projections extending from the inner surface of a distal end thereof to form a needle guide configured to hold the needle shaft in place.

16. The assembly of claim 15, wherein the plurality of projections comprises at least two needle-stabilizing projections disposed on either side of the needle shaft and offset from one another along a longitudinal axis of the needle shaft, each of the at least two needle-stabilizing projections having a sloped surface abutting the needle shaft.

17. The assembly of any one of claims 11-16, wherein the first contact surface is disposed along the perimeter of a distal end of the foot, and the second contact surface is substantially centrally disposed at the distal end of the housing.

18. The assembly of any one of claims 11-17, wherein the first housing portion and the second housing portion are of at least substantially similar or identical construction.

19. The assembly of any one of claims 11-18, wherein the housing is generally cylindrical in shape.

20. The assembly of any one of claims 11-19, wherein each of the first housing portion and the second housing portion is generally semi-cylindrical in shape.

21. The assembly of any one of claims 11-20, wherein the friction means comprises at least two protrusions extending from an inner surface of a proximal end of the foot being in contact with at least two corresponding grooves located on an outer surface of the distal end of the housing of the needle adaptor body, wherein a radial dimension defined by the at least two protrusions before assembly of the needle adaptor body and the foot, is smaller than a radial dimension defined by the at least two corresponding grooves, the static friction being provided by radial clamping.

22. The assembly of claim 21, wherein the at least two corresponding grooves are configured to prevent disengagement of the foot from the needle adaptor body by limiting movement of the foot away from the needle adaptor body following engagement of the at least two protrusions extending from the inner surface of the proximal end of the foot with the at least two corresponding grooves.

23. The assembly of claim 21 or 22, wherein the at least two corresponding grooves are oriented generally parallel to a longitudinal axis of the housing.

24. The assembly of claim 23, further comprising at least two deactivation grooves located on the outer surface of the distal end of the housing of the needle adaptor body,
wherein each of the at least two deactivation grooves intersects one of the at least two corresponding grooves at an angle relative to the longitudinal axis of the housing, such that axial movement of the foot away from the needle adaptor body and rotation of the foot relative to the needle adaptor body engages the at least two protrusions with the at least two deactivation grooves,
wherein each of the at least two deactivation grooves comprises an indentation complementary to a shape of each of the at least two protrusions to fixedly engage each of the at least two protrusions, such that the needle adaptor body is held in a fixed, deactivated position relative to the foot,
wherein the first end of the needle shaft does not extend beyond the first contact surface when the needle adaptor body is in the fixed, deactivated position relative to the foot.

25. The assembly of any one of claims 11-24, further comprising a locking mechanism for providing a locked mode and an unlocked mode of the device,
the locked mode being a mode of the assembly, wherein the needle adaptor body is prevented from moving axially towards the foot, even when an axial force larger than the predefined static friction is exerted on the needle adaptor body relative to the foot;
the unlocked mode being a mode of the assembly wherein the needle adaptor body is allowed to move towards the foot, when an axial force larger than the predefined static friction is applied to the needle adaptor body relative to the foot.

26. The assembly of claim 25, wherein the locking mechanism comprises a removable safety clip configured to engage with a portion of the outer surface of the housing to maintain the foot and needle adaptor body spaced apart from one another to prevent the needle adaptor body from moving axially towards the foot.

27. A method for assembling the needle adaptor of claim 1, the method comprising:
obtaining the first housing portion and the second housing portion;
obtaining the needle unit;
optionally, measuring a length of the needle shaft, and removing a preselected portion of a distal end of each of the first housing portion and the second housing portion based on the length of the needle shaft;
mounting the needle unit in one of the first housing portion and the second housing portion by inserting the at least a portion of one or both of the pair of radially extending diametrically opposing flanges of the needle unit into the gap formed between the at least two consecutive transverse walls or projections; and
engaging the first housing portion and the second housing portion with one another to form the housing.

28. A method for assembling the needle adaptor of claim 2, the method comprising:
obtaining the first housing portion and the second housing portion;
obtaining the needle unit;
measuring a length of the needle shaft;
determining whether the needle unit is to be mounted in the first orientation or the second orientation based on the length of the needle shaft;
optionally, removing a preselected portion of a distal end of each of the first housing portion and the second housing portion based on the length of the needle shaft and based on whether the needle unit is to be mounted in the first orientation or the second orientation;
mounting the needle unit in one of the first housing portion and the second housing portion in the first orientation or the second orientation by inserting the at least a portion of one or both of the pair of radially extending diametrically opposing flanges of the needle unit into the gap formed between the at least two consecutive transverse walls or projections; and
engaging the first housing portion and the second housing portion with one another to form the housing.

29. The method of claim 27 or 28, wherein the at least a portion of one or both of the pair of radially extending diametrically opposing flanges of the needle unit has a frictional engagement with opposing surfaces of the at least two consecutive transverse walls or projections of each of the first housing portion and the second housing portion when received in the gap therebetween.

30. The method of any one of claims 27-29, wherein the first housing portion and the second housing portion are configured for snap-fit engagement with one another to form the housing.

31. The method of any one of claims 27-30, wherein each of the first housing portion and the second housing portion further comprises a plurality of projections extending from the inner surface of a distal end thereof to form a needle guide configured to hold the needle shaft in place.

32. The method of claim 31, wherein the plurality of projections comprises at least two needle-stabilizing projections disposed on either side of the needle shaft and offset from one another along a longitudinal axis of the needle shaft, each of the at least two needle-stabilizing projections having a sloped surface that abuts the needle shaft when the needle unit is mounted in one of the first housing portion and the second housing portion.

33. The method of any one of claims 27-32, wherein the first contact surface is disposed along the perimeter of the distal end of the housing, and the second contact surface is substantially centrally disposed at the distal end of the housing.

34. The method of any one of claims 27-33, wherein the first housing portion and the second housing portion are of at least substantially similar or identical construction.

35. The method of any one of claims 27-34, wherein the housing is generally cylindrical in shape.

36. The method of any one of claims 27-35, wherein each of the first housing portion and the second housing portion is generally semi-cylindrical in shape.

37. The method of any one of claims 27-36, wherein removing the preselected portion of the distal end of each of the first housing portion and the second housing portion comprising cutting the preselected portion of the distal end of each of the first housing portion and the second housing portion, such as by laser cutting.

38. The method of any one of claims 27-37, wherein the method is automated.

39. A method for assembling the assembly of claim 11, wherein the assembly optionally further comprises a locking mechanism comprising a removable safety clip configured to engage with a portion of the outer surface of the housing to maintain the foot and needle adaptor body spaced apart from one another to prevent the needle adaptor body from moving axially towards the foot, the method comprising:
obtaining the foot;
obtaining the first housing portion and the second housing portion forming the housing of the needle adaptor body;
obtaining the needle unit;
optionally, obtaining the removable safety clip;
optionally, measuring a length of the needle shaft, and removing a preselected portion of a distal end of each of the first housing portion and the second housing portion based on the length of the needle shaft;
mounting the needle unit in one of the first housing portion and the second housing portion by inserting the at least a portion of one or both of the pair of radially extending diametrically opposing flanges of the needle unit into the gap formed between the at least two consecutive transverse walls or projections;
engaging the first housing portion and the second housing portion with one another to form the housing of the needle adaptor body;
engaging the removable safety clip, if present, with the portion of the outer surface of the housing; and
engaging the foot and the needle adaptor body.

40. A method for assembling the assembly of claim 12, wherein the assembly optionally further comprises a locking mechanism comprising a removable safety clip configured to engage with a portion of the outer surface of the housing to maintain the foot and needle adaptor body spaced apart from one another to prevent the needle adaptor body from moving axially towards the foot, the method comprising:
obtaining the foot;
obtaining the first housing portion and the second housing portion forming the housing of the needle adaptor body;
obtaining the needle unit;
optionally, obtaining the removable safety clip;
measuring a length of the needle shaft;
determining whether the needle unit is to be mounted in the first orientation or the second orientation based on the length of the needle shaft;
optionally, removing a preselected portion of a distal end of each of the first housing portion and the second housing portion based on the length of the needle shaft and based on whether the needle unit is to be mounted in the first orientation or the second orientation;
mounting the needle unit in one of the first housing portion and the second housing portion in the first orientation or the second orientation by inserting the at least a portion of one or both of the pair of radially extending diametrically opposing flanges of the needle unit into the gap formed between the at least two consecutive transverse walls or projections; and
engaging the first housing portion and the second housing portion with one another to form the housing of the needle adaptor body;
engaging the removable safety clip, if present, with the portion of the outer surface of the housing; and
engaging the foot and the needle adaptor body.

41. The method of claim 39 or 40, wherein the at least a portion of one or both of the pair of radially extending diametrically opposing flanges of the needle unit has a frictional engagement with opposing surfaces of the at least two consecutive transverse walls or projections of each of the first housing portion and the second housing portion when received in the gap therebetween.

42. The method of any one of claims 39-41, wherein the first housing portion and the second housing portion are configured for snap-fit engagement with one another to form the housing.

43. The method of any one of claims 39-42, wherein each of the first housing portion and the second housing portion further comprises a plurality of projections extending from the inner surface of a distal end thereof to form a needle guide configured to hold the needle shaft in place.

44. The method of claim 43, wherein the plurality of projections comprises at least two needle-stabilizing projections disposed on either side of the needle shaft and offset from one another along a longitudinal axis of the needle shaft, each of the at least two needle-stabilizing projections having a sloped surface abutting the needle shaft.

45. The method of any one of claims 39-44, wherein the first contact surface is disposed along the perimeter of a distal end of the foot, and the second contact surface is substantially centrally disposed at the distal end of the housing.

46. The method of any one of claims 39-45, wherein the first housing portion and the second housing portion are of at least substantially similar or identical construction.

47. The method of any one of claims 39-46, wherein the housing is generally cylindrical in shape.

48. The method of any one of claims 39-47, wherein each of the first housing portion and the second housing portion is generally semi-cylindrical in shape.

49. The method of any one of claims 39-48, wherein the friction means comprises at least two protrusions extending from an inner surface of a proximal end of the foot being in contact with at least two corresponding grooves located on an outer surface of the distal end of the housing of the needle adaptor body, wherein a radial dimension defined by the at least two protrusions before assembly of the needle adaptor body and the foot, is smaller than a radial dimension defined by the at least two corresponding grooves, the static friction being provided by radial clamping;
wherein engaging the foot and the needle adaptor body comprises engaging the at least two protrusions extending from the inner surface of the proximal end of the foot with the at least two corresponding grooves located on the outer surface of the distal end of the housing of the needle adaptor body.

50. The method of claim 49, wherein the at least two corresponding grooves are configured to prevent disengagement of the foot from the needle adaptor body by limiting movement of the foot away from the needle adaptor body following engagement of the at least two protrusions extending from the inner surface of the proximal end of the foot with the at least two corresponding grooves.

51. The method of claim 49 or 50, wherein the at least two corresponding grooves are oriented generally parallel to a longitudinal axis of the housing.

52. The method of claim 51, wherein the assembly further comprises at least two deactivation grooves located on the outer surface of the distal end of the housing of the needle adaptor body,
wherein each of the at least two deactivation grooves intersects one of the at least two corresponding grooves at an angle relative to the longitudinal axis of the housing, such that axial movement of the foot away from the needle adaptor body and rotation of the foot relative to the needle adaptor body engages the at least two protrusions with the at least two deactivation grooves,
wherein each of the at least two deactivation grooves comprises an indentation complementary to a shape of each of the at least two protrusions to fixedly engage each of the at least two protrusions, such that the needle adaptor body is held in a fixed, deactivated position relative to the foot,
wherein the first end of the needle shaft does not extend beyond the first contact surface when the needle adaptor body is in the fixed, deactivated position relative to the foot.

53. The method of any one of claims 39-52, wherein removing the preselected portion of the distal end of each of the first housing portion and the second housing portion comprising cutting the preselected portion of the distal end of each of the first housing portion and the second housing portion, such as by laser cutting.

54. The method of any one of claims 39-53, wherein the method is automated.

55. A method of administering a fluid to a subject via injection, the method comprising:
(a) obtaining the needle adaptor of any one of claims 1-10;
(b) obtaining a syringe or other dosing device, wherein the other dosing device comprises a dispensing tip that is similar in size and shape to a syringe tip,
wherein the syringe or the other dosing device is loaded with the fluid to be administered to the subject;
(c) inserting the tip of the syringe or the other dosing device into the channel disposed at the proximal end of the housing so as to engage the tip with the needle hub;
(d) engaging the first contact surface with the skin of the subject;
(e) pushing the housing against the skin to allow the first end of the needle shaft to penetrate the skin;
(f) expelling the fluid from the syringe or the other dosing device through the needle shaft into the subject; and
(g) optionally, engaging the needle adaptor with a safety holder, wherein the safety holder has an open end for receiving at least the distal end of the needle adaptor housing and a closed end, the closed end comprising opposed wings for stabilizing the safety holder on a horizontal surface.

56. A method of administering a fluid to a subject via injection, the method comprising:
(a) obtaining the assembly of any one of claims 11-26, wherein the needle adaptor body is in the first position;
(b) obtaining a syringe or other dosing device, wherein the other dosing device comprises a dispensing tip that is similar in size and shape to a syringe tip,
wherein the syringe or the other dosing device is loaded with the fluid to be administered to the subject;
(c) inserting the tip of the syringe or the other dosing device into the channel disposed at the proximal end of the needle adaptor housing so as to engage the tip with the needle hub;
(d) engaging the first contact surface of the foot with the skin of the subject;
(e) pushing the housing of the needle adaptor body towards the foot to move the needle adaptor body from the first position to the second position, thus causing the first end of the needle shaft to penetrate the skin; and
(f) expelling the fluid from the syringe or the other dosing device through the needle shaft into the subject.

57. A method of administering a fluid to a subject via injection, the method comprising:
(a) obtaining the assembly of claim 24, wherein the needle adaptor body is in the first position;
(b) obtaining a syringe or other dosing device, wherein the other dosing device comprises a dispensing tip that is similar in size and shape to a syringe tip,
wherein the syringe or the other dosing device is loaded with the fluid to be administered to the subject;
(c) inserting the tip of the syringe or the other dosing device into the channel disposed at the proximal end of the needle adaptor housing so as to engage the tip with the needle hub;
(d) engaging the first contact surface of the foot with the skin of the subject;
(e) pushing the housing of the needle adaptor body towards the foot in an axial direction to move the needle adaptor body from the first position to the second position, thus causing the first end of the needle shaft to penetrate the skin;
(f) expelling the fluid from the syringe or the other dosing device through the needle shaft into the subject; and
(g) pulling the housing of the needle adaptor body away from the foot in an axial direction and rotating the foot relative to the needle adaptor body to engage the at least two protrusions with the at least two deactivation grooves and to fixedly engage each of the at least two protrusions in the indentation in each of the at least two deactivation grooves, such that the needle adaptor body is held in the fixed, deactivated position relative to the foot.

58. A method of administering a fluid to a subject via injection, the method comprising:
(a) obtaining the assembly of claim 26, wherein the needle adaptor body is in the first position;
(b) obtaining a syringe or other dosing device, wherein the other dosing device comprises a dispensing tip that is similar in size and shape to a syringe tip,
wherein the syringe or the other dosing device is loaded with the fluid to be administered to the subject;
(c) inserting the tip of the syringe or the other dosing device into the channel disposed at the proximal end of the needle adaptor housing so as to engage the tip with the needle hub;
(d) engaging the first contact surface of the foot with the skin of the subject;
(e) pushing the housing of the needle adaptor body towards the foot in an axial direction to move the needle adaptor body from the first position to the second position, thus causing the first end of the needle shaft to penetrate the skin;
(f) expelling the fluid from the syringe or the other dosing device through the needle shaft into the subject; and
(g) pulling the housing of the needle adaptor body away from the foot in an axial direction and rotating the foot relative to the needle adaptor body to engage the at least two protrusions with the at least two deactivation grooves and to fixedly engage each of the at least two protrusions in the indentation in each of the at least two deactivation grooves, such that the needle adaptor body is held in the fixed, deactivated position relative to the foot;
the method further comprising removing the safety clip from the outer surface of the housing after step (c) and prior to step (d), or after step (d) and prior to step (e).

59. A kit comprising:
the needle adaptor of any one of claims 1-10;
a syringe or other dosing device, wherein the other dosing device comprises a dispensing tip that is similar in size and shape to a syringe tip, wherein the syringe or the other dosing device is optionally loaded with the fluid to be administered to the subject;
optionally, a vial containing the fluid to be administered to the subject;
optionally, a removable needle unit or other means for extracting the fluid from the optional vial into the syringe or the other dosing device, the removable needle unit being removable for allowing the tip of the syringe or the other dosing device to be inserted into the channel of the housing;
optionally, a safety holder, wherein the safety holder has an open end for receiving at least the distal end of the needle adaptor housing and a closed end, the closed end comprising opposed wings for stabilizing the safety holder on a horizontal surface; and
optionally, instructions for use.

60. A kit comprising:
the assembly of any one of claims 11-26;
a syringe or other dosing device, wherein the other dosing device comprises a dispensing tip that is similar in size and shape to a syringe tip, wherein the syringe or the other dosing device is optionally loaded with the fluid to be administered to the subject;
optionally, a vial containing the fluid to be administered to the subject;
optionally, a removable needle unit or other means for extracting the fluid from the optional vial into the syringe or the other dosing device, the removable needle unit being removable for allowing the tip of the syringe or the other dosing device to be inserted into the channel of the housing; and
optionally, instructions for use.

61. An injection device comprising:
the needle adaptor of any one of claims 1-10; and
a syringe or other dosing device, wherein the other dosing device comprises a dispensing tip that is similar in size and shape to a syringe tip, wherein the syringe or the other dosing device is optionally loaded with a fluid to be administered to a subject.

62. An injection device comprising:
the assembly of any one of claims 11-26; and
a syringe or other dosing device, wherein the other dosing device comprises a dispensing tip that is similar in size and shape to a syringe tip, wherein the syringe or the other dosing device is optionally loaded with a fluid to be administered to a subject.
